(19) United States
(12) Patent Application Publication
(10) Pub. No.: US 2024/0029901 A1
(43) Pub. Date: Jan. 25, 2024

(54) Systems and Methods to generate a personalized medical summary (PMS) from a practitioner-patient conversation.

(71) Applicants: Matvey Ezhov, Yerevan (AM); Alex Sanders, Tel Aviv (IL)

(72) Inventors: Matvey Ezhov, Yerevan (AM); Alex Sanders, Tel Aviv (IL)

(21) Appl. No.: 18/225,486

(22) Filed: Jul. 24, 2023

Related U.S. Application Data
(63) Continuation-in-part of application No. 17/456,802, filed on Nov. 29, 2021.
Application No. 17/456,802 is a continuation-in-part of application No. 16/847,563, filed on Apr. 13, 2020, now Pat. No. 11,464,467.
Application No. 16/847,563 is a continuation-in-part of application No. 16/783,615, filed on Feb. 6, 2020, now Pat. No. 11,443,423.
Application No. 16/783,615 is a continuation-in-part of application No. 16/175,067, filed on Oct. 30, 2018, now Pat. No. 10,991,091.

(51) Int. Cl. G16H 80/00; G16H 50/20

(52) U.S. Cl. CPC G16H 80/00; G16H 50/20

(57) ABSTRACT

The invention relates to a method to generate a personalized medical summary (PMS) from a practitioner-patient conversation capturing a conversation between a practitioner and a patient, transcribing the conversation between the practitioner and the patient and generating the PMS based on the transcribed conversation.

### BACKGROUND

### Field

This invention relates generally to medical diagnostics and communication, and more particularly, the invention concerns generating a personalized medical summary from a practitioner-patient conversation.

### Related Art

Modern image generation systems play an important role in disease detection and treatment planning. Few existing systems and methods were discussed as follows. One common method utilized is dental radiography, which provides dental radiographic images that enable the dental professional to identify many conditions that may otherwise go undetected and to see conditions that cannot be identified clinically. Another technology is cone beam computed tomography (CBCT) that allows to view structures in the oral-maxillofacial complex in three dimensions. Hence, cone beam computed tomography technology is most desired over the dental radiography.

However, CBCT includes one or more limitations, such as time consumption and complexity for personnel to become fully acquainted with the imaging software and correctly using digital imaging and communications in medicine (DICOM) data. American Dental Association (ADA) also suggests that the CBCT image should be evaluated by a dentist with appropriate training and education in CBCT interpretation. Further, many dental professionals who incorporate this technology into their practices have not had the training required to interpret data on anatomic areas beyond the maxilla and the mandible. To address the foregoing issues, deep learning has been applied to various medical imaging problems to interpret the generated images, but its use remains limited within the field of dental radiography. Further, most applications only work with 2D X-ray images.

Therefore, there is a need for an automated parsing pipeline system and method for anatomical localization and condition classification, with minimal image analysis training and visual ambiguities. Furthermore, there is a need for applying deep learning models for constructing panoramas from CBCT images that emphasize Elements of Interest (EoI) for more defined and actionable imaging. Going a step further, there is a need for further processing these EoI focused panoramas using deep learning methods to generate accurate 3D teeth segmentation masks with localization.

Furthermore, extant annotation tools configured for a full mouth set of x-rays (FMX) or panoramic radiographs enable overlaying images with descriptive information for centralized storage and efficient querying support, allowing practitioners and proxy to construct complex queries to find meaningful, related cases efficiently later. The extant annotation tools do not support localizing and enumerating teeth in the images using neural networks, and certainly, do not support sorting images into a full mount table with neural network-mediated classification of a tooth condition with diagnostic value. Therefore, there is void in the art for an automated localization, numeration, and diagnostic system and method for FMX and panoramic images for improved dental health outcomes.

Further yet, there is a need for user interface functionality involving unique visual elements, routines, and cursor controls that enable quick-glance analysis across analytic/diagnostic layers of information.

Further yet, once, the patient receives the diagnosis, clear and effective communication of a diagnosis to a patient is critical to ensure understanding, emotional support, treatment adherence, empowerment, and trust. Effective communication of a diagnosis to a patient is crucial for several reasons: (1) Understanding: It is essential for patients to understand their diagnosis so that they can make informed decisions about their treatment options. A clear diagnosis communication helps the patient understand their condition, its severity, and the likely outcome, (2) Emotional impact: Receiving a diagnosis can be overwhelming and emotional for patients. Clear communication can help alleviate anxiety and provide emotional support, (3) Treatment adherence: A patient who understands their diagnosis is more likely to adhere to their treatment plan. If a patient does not understand their diagnosis, they may not understand the importance of their treatment or follow through with it, (4) Empowerment: When a patient is involved in the diagnosis communication process, they feel more empowered to participate in their treatment decisions. Empowered patients are more likely to actively participate in their care, leading to better health outcomes, and (5) Trust: Effective communication of a diagnosis builds trust between the patient and healthcare provider. Patients who feel heard and respected are more likely to have a positive view of their healthcare provider and follow their recommendations.

Further yet, the practice of medicine involves complex-often stressful communication between healthcare clinicians and patients and their families. Good communication between clinicians and patients is essential to enable good outcomes and avoid medical/dental errors. Sometimes patients cannot express their concerns and needs clearly. Conversely, clinicians often overestimate their communication skills, and such skills have been shown to decline during a physician's career. Breakdown in communication can lead to harm and suboptimal treatment. It is of utmost importance of involving the patient as a partner in the treatment and planning process, something that can only occur with good clinician-patient discourse. Poor communication can lead to a medical error when a patient does not report their allergies or health history to a clinician, or when a clinician does not correctly or thoroughly record a medical history or medication list in patient's case. When clinicians do not communicate well with each other, errors can occur because of incorrect or missing information. In the dental/medical space, technological advancements have been long a driving force behind improving patient care and outcomes. AI has the potential to revolutionize communication in medicine by providing clinicians with personalized, highly detailed assessments of their communication skills.

The use of Artificial Intelligence (AI) can significantly improve practitioner-patient communication in various ways. Some examples include, but are limited to: (1) Personalized queries: AI-powered chatbots and assistants can generate personalized queries based on the patient's medical history, dental records, and specific symptoms. This can help practitioners gather relevant information quickly and efficiently, leading to better diagnosis and treatment planning. (2) Simplified language: AI can help simplify complex medical terminology and jargon into language that patients can understand. This can help patients better understand their condition and treatment options, leading to better compliance and improved outcomes. (3) 24/7 availability: AI-powered chatbots and assistants can be available 24/7, allowing patients to ask questions and receive information outside of regular office hours. This can improve patient satisfaction and engagement. (4) Consistent messaging: AI can ensure that patients receive consistent messaging and information across different communication channels (such as phone, email, and text). This can help avoid confusion and ensure that patients receive accurate and reliable information. (5) Remote consultations: AI-powered communication tools can enable remote consultations between practitioners and patients, allowing for convenient and efficient communication without the need for in-person visits. This can improve access to care, particularly for patients who live in remote or underserved areas. Overall, the use of AI in practitioner-patient communication has the potential to significantly improve patient outcomes and satisfaction, while also making the work of dental practitioners more efficient and effective.

A US Patent Application, US 2008/0091631, provides a computer software medical diagnosis system with which users can interact over the Internet using a web browser in order to obtain a medical diagnosis or recommendation. However, the application fails to take into account a clinician expert opinion and a human touch while diagnosing a patient. The human touch is unconditionally essential when it comes to diagnosing dental/medical issues and developing treatment plans. Hence, it's has become apparent that there is a need for an AI-aided system to deliver practitioner-patient communication.

Chart reviews and medical record summaries are essential tools for healthcare providers and attorneys. They can be used to improve the quality of care, protect patients, and support research. Chart reviews involves analyzing medical records to identify areas where care can be improved, while medical record summaries provide a clear and concise overview of a patient's medical history and treatment plan. Both chart review and medical record summaries can be used to provide legal defense in the event of a lawsuit, facilitate communication between healthcare providers and patients, help patients understand their medical condition and treatment plan, support claims for insurance reimbursement, and research new medical treatments and procedures.

Additionally, a medical summary prepared by a doctor after a patient visit holds significant importance as it ensures continuity of care by providing a concise overview of the patient's medical history, current health status, and treatment plan. It serves as a communication tool among healthcare providers, facilitates reference for future visits, aids in collaboration with other professionals, empowers patients with knowledge, and serves as a legal document for documentation purposes. It plays a vital role in effective healthcare delivery by promoting clear communication, continuity of care, and comprehensive documentation of the patient's medical information.

While preparing a medical summary offers several advantages, it also poses potential disadvantages. These include the omission of important details, the risk of misinterpretation or miscommunication, limited contextual information, privacy and security concerns, the potential for bias or subjective interpretation, and the time constraints and administrative burden it places on healthcare providers—not including the time put in by the practitioners. It is crucial to be aware of these drawbacks and take steps to address them through effective communication, thorough documentation, and maintaining privacy standards during the preparation of medical summaries. Therefore, there is a void in the market for a generation of an AI-driven personalized medical summary that will drastically improve practitioner-patient communication and significantly reduce time constraints and administrative burdens on practitioners.

Proper medical record documentation is essential for providing quality care to patients, protecting patients from harm, and ensuring that patients receive the care they need. However, it can be a time-consuming and tedious process. An AI-aided system could help to streamline the process and ensure that medical records are accurate and complete. Medical records also tell the patient's “story,” the presenting problem and the treatment received, helps to plan and evaluate a patient's treatment, and creates a permanent record for the patient's future care. An AI-aided system could also help to improve communication between healthcare providers and patients thus, leading to better decision-making and improved outcomes. In addition, an AI-aided system could help to reduce risk management exposure. By identifying potential risks and hazards, the system could help to prevent medical errors and lawsuits.

Thus, an AI-aided system could have a significant impact on the healthcare industry by improving the quality of medical record documentation, communication, and risk management, the system could help to improve patient care and outcomes.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. Embodiments disclosed include an automated parsing pipeline system and method for anatomical localization and condition classification. Embodiments disclosed also include for a method and system for constructing a panorama with elements of interest (EoI) emphasized of a teeth arch or any point of interest in an oral-maxillofacial complex. Further embodiments disclosed include for an automated system and method for localizing, enumerating, and diagnosing a tooth/tooth condition from a FMX/Panoramic image for improved dental outcomes.

In an embodiment, the system comprises an input event source, a memory unit in communication with the input event source, a processor in communication with the memory unit, a volumetric image processor in communication with the processor, a voxel parsing engine in communication with the volumetric image processor and a localizing layer in communication with the voxel parsing engine. In one embodiment, the memory unit is a non-transitory storage element storing encoded information. In one embodiment, at least one volumetric image data is received from the input event source by the volumetric image processor. In one embodiment, the input event source is a radio-image gathering source.

The processor is configured to parse the at least one received volumetric image data into at least a single image frame field of view by the volumetric image processor. The processor is further configured to localize anatomical structures residing in the at least single field of view by assigning at least one of a pixel and voxel a distinct anatomical structure by the voxel parsing engine. In one embodiment, the single image frame field of view is pre-processed for localization, which involves rescaling using linear interpolation. The pre-processing involves use of any one of a normalization schemes to account for variations in image value intensity depending on at least one of an input or output of volumetric image. In one embodiment, localization is achieved using any one of a fully convolutional network (FCN) or plain classification convolutional neural network (CNN).

The processor is further configured to select at least one of all pixels and voxels (p/v) belonging to the localized anatomical structure by finding a minimal bounding rectangle around the p/v and the surrounding region for cropping as a defined anatomical structure by the localization layer. The bounding rectangle extends equally in all directions to capture the tooth and surrounding context. In one embodiment, the automated parsing pipeline system further comprises a detection module. The processor is configured to detect or classify the conditions for each defined anatomical structure within the cropped image by a detection module or classification layer. In one embodiment, the classification is achieved using any one of a fully convolutional network or plain classification convolutional neural network (FCN/CNN).

In another embodiment, an automated parsing pipeline method for anatomical localization and condition classification is disclosed. At one step, at least one volumetric image data is received from an input event source by a volumetric image processor. At another step, the received volumetric image data is parsed into at least a single image frame field of view by the volumetric image processor. At another step, the single image frame field of view is pre-processed by controlling image intensity value by the volumetric image processor. At another step, the anatomical structure residing in the single pre-processed field of view is localized by assigning each p/v a distinct anatomical structure ID by the voxel parsing engine. At another step, all p/v belonging to the localized anatomical structure is selected by finding a minimal bounding rectangle around the voxels and the surrounding region for cropping as a defined anatomical structure by the localization layer. In another embodiment, the method includes a step of, classifying the conditions for each defined anatomical structure within the cropped image by the classification layer.

Further embodiments disclosed include a method and system for constructing a panorama with elements of interest (EoI) emphasized of a teeth arch or any point of interest in an oral-maxillofacial complex. Other embodiments of this aspect also include for any various deep learning models or modules for processing any one of the steps in the construction of 2D or 3D or 2D/3D-fused teeth segmentation masks with localization. It should be appreciated that any point of interest in the oral-maxillofacial complex may be translated into the EoI-focused panorama/mask for higher-defined actionable imaging.

Further embodiments disclose for a system and method for localizing, annotating, and diagnosing a tooth/tooth condition from a FMX or panoramic image. In one embodiment, the system may comprise an image processor; a localization layer; a sorting engine; a processor; a non-transitory storage element coupled to the processor; encoded instructions stored in the non-transitory storage element, wherein the encoded instructions when implemented by the processor, configure the system to: receive a series of at least one of an intra-oral or panoramic images constituting a full mouth series from a radio-image gathering or digital capturing source for processing by the image processor; parse the series of images into at least a single image frame field of view by said image processor; localize and enumerate at least one tooth residing in the at least single image frame field of view by assigning each pixel a distinct tooth structure by selecting all pixels belonging to the localized tooth structure by finding a minimal bounding rectangle around said pixels and the surrounding region for cropping as a defined enumerated tooth structure image by the localization layer; and sort images using the defined enumerated tooth structure images to fill an FMX mounting table by the sorting engine.

In another embodiment, the system may further comprise an image processor; a localization layer; a diagnostic module or classification layer; a processor; a non-transitory storage element coupled to the processor; encoded instructions stored in the non-transitory storage element, wherein the encoded instructions when implemented by the processor, configure the system to: receive a series of at least one of an intra-oral or panoramic images constituting a full mouth series from a radio-image gathering or digital capturing source for processing by the image processor; localize and enumerate at least one tooth residing in at least single image frame field of view by assigning each pixel a distinct tooth structure by selecting all pixels belonging to the localized tooth structure by finding a minimal bounding rectangle around said pixels and the surrounding region for cropping as a defined enumerated tooth structure image by the localization layer; and detect conditions for each defined enumerated tooth structure within a cropped image, wherein conditions are detected by the classification layer, wherein the classification layer at least one of detects or segments conditions and pathologies on at least one of the enumerated tooth structures within the cropped image.

In yet another embodiment, a method is disclosed, entailing the steps involved for localizing, annotating, and optionally, diagnosing a tooth condition carried out by the automated pipeline or system. Generally, the steps are: receiving a series of at least one of an intra-oral or panoramic images constituting a full mouth series from a radio-image gathering or digital capturing source for processing; localizing and enumerating at least one tooth residing in at least single image frame field of view by assigning each pixel a distinct tooth structure by selecting all pixels belonging to the localized tooth structure by finding a minimal bounding rectangle around said pixels and the surrounding region for cropping as a defined enumerated tooth structure image; and (optionally) sorting images using the defined enumerated tooth structure images to fill an FMX mounting table.

Furthermore, the method may optionally further comprise the step of detecting conditions for each defined enumerated tooth structure within a cropped image, wherein conditions are detected by at least one of detecting or segmenting conditions and pathologies on at least one of the enumerated tooth structures within the cropped image. Other embodiments of this aspect include corresponding computer systems, apparatus, and computer programs recorded on one or more computer storage devices, each configured to perform the actions of the methods.

Further embodiments disclose a system and method for receiving at least one image frame; localizing at least one of a present tooth, dental, or non-dental condition inside the received image frame and identify it by at least one of a number, name, or short-hand; extracting the at least one identified tooth, dental, or non-dental condition within the received image; classifying the at least one tooth, dental, or non-dental condition based on the extracted; and representing results of the at least one classified area of interest in at least one of three layers, wherein the layers are an image-based layer, infographic-based layer, or an informational layer. In another embodiment, a visual report module generates novel visual elements, routines, and cursor controls that enable quick glance analysis across analytic/diagnostic layers of imaging data.

Further embodiments disclose a system and method to generate a personalized medical summary (PMS) from a practitioner-patient conversation comprising capturing a conversation between the practitioner and the patient, transcribing the conversation between the practitioner and the patient and generating the PMS based on the transcribed conversation. The conversations between practitioner-patient are captured via a recording device. The recording device is at least one of voice recorders, smart phones, smart & digital devices, microphones, cameras, audio or video recorder, PC, and digital transcription devices. Further yet, in an embodiment of the invention comprising transcribing the practitioner-patient conversation into a textual transcription using an automated speech recognition (ASR), wherein the ASR at least one of, an off-the shelf, custom-built or a third-party service. Additionally, achieved by extracting clinically relevant information using at least one of a general-purpose large language model (LLM), a fine-tuned LLM trained to transcribe medical conversation or a custom-built LLM.

Further yet in an embodiment of the invention, a system to generate a personalized medical summary (PMS) from practitioner-patient conversations comprising a processor, a diagnosis-AI module (DAIM), a non-transitory storage element coupled to the processor over a network, encoded instructions stored in the non-transitory storage element, wherein the encoded instructions when implemented by the processor, configure the system to capture a conversation between the practitioner and the patient, transcribe the conversation between the practitioner and the patient and generate the PMS for the patient based on the transcribed conversation via the DAIM. The conversations between practitioner-patient are captured via a recording device. The recording device is at least one of voice recorders, smart phones, smart & digital devices, microphones, cameras, audio or video recorder, PC, and digital transcription devices. Further yet, in an embodiment of the invention comprising transcribing the practitioner-patient conversation into a textual transcription using an automated speech recognition (ASR), wherein the ASR at least one of, an off-the shelf, custom-built or a third-party service and the ASR is performed by at least one of acoustic modeling-based ASR or neural network-based ASR. Further yet, the PMS is generated by a diagnosis-AI module (DAIM) by extracting clinically relevant information from the practitioner-patient conversation and is rendered using at least one of a template-filling, do-by-example, and free-form summary format.

In yet another embodiment of the invention, a method to generate a personalized medical summary (PMS) from practitioner-patient conversation comprising capturing a conversation between the practitioner and the patient, transcribing the conversation between the practitioner and the patient and generating the PMS for the patient based on the transcribed conversation, wherein the PMS is generated by a dental diagnosis module (DAIM) using diagnosis-AI module (DAIM).

Further embodiments disclose a system and method to a method to deliver a dental practitioner-patient conversation, said method comprising: receiving a patient data and a practitioner prompt; generating a practitioner first query for a patient based on the received patient data and the practitioner prompt and generating at least one of a practitioner second query or a diagnosis for the patient based on the patient response to the practitioner first query to enhance practitioner-patient communication. Further yet, the patient data is at least one of current patient condition, patient dental/medical history, physical and mental health, dental/medical treatments, X-rays/scans, medical complaints and list of medication. Additionally, in an embodiment of the invention, wherein the query is based on at least one of a diagnosis, treatment, planning, and follow-up of the dental/medical procedures for the patient.

In a preferred embodiment of the invention, the method commences by collecting relevant information about the patient and the practitioner prompt that will guide the conversation. This information could include the patient's age, medical history, dental problems, and the practitioner's preferences. Further yet, based on the patient data and the practitioner prompt, the AI system generates a dental query that the practitioner would likely ask the patient. This query is generated using a dental diagnosis module (DAIM) that has been optimized using reinforcement learning. Reinforcement learning involves training the DAIM to make decisions based on a reward signal, which represents how well its decisions align with some desired outcome. In the context of dental diagnosis, the reward signal might represent factors like patient satisfaction, cost-effectiveness, and adherence to best practices. In another embodiment, the DAIM may be trained using a combination of supervised fine-tuning and reinforcement learning to improve the accuracy of the generated query. Additionally, once the patient responds to the practitioner first query, the DAIM generates either a practitioner second query or a dental diagnosis, depending on the specific needs of the consultation. This helps to enhance the communication between the practitioner and the patient, as the DAIM can provide valuable insights and recommendations based on the patient's responses.

Further embodiments of the invention disclose a method to deliver a practitioner-patient conversation, said method comprising: receiving patient data and a practitioner prompt; generating a practitioner first query for a patient based on the received patient data and the practitioner prompt; and generating at least one of a practitioner second query or a diagnosis for the patient based on the response to the practitioner first query to enhance practitioner-patient communication.

Additionally in further embodiments of the invention, the method commences by receiving data about the patient and a prompt from the practitioner. This data could include information about the patient's medical history, current symptoms, and any relevant test results, while the prompt could be a question or suggestion from the practitioner about how to proceed with the diagnosis or treatment. Using the received patient data and the practitioner prompt, the method generates a practitioner first query for the patient. This query is designed to elicit more information from the patient, clarify any uncertainties or ambiguities in the data, and guide the practitioner in making an accurate diagnosis. Based on the response to the practitioner first query by the patient, the method generates either a practitioner second query or a diagnosis for the patient. The second query could be another question or clarification to help the practitioner narrow down the diagnosis further, while the diagnosis could be a preliminary or final conclusion about the patient's condition.

Further embodiments of the invention discloses a system to deliver a doctor-patient conversation, comprising: a dental diagnosis module (DAIM), a processor, a non-transitory storage element coupled to the processor over a network, encoded instructions stored in the non-transitory storage element, wherein the encoded instructions when implemented by the processor, configure the system, to receiving a patient data and a practitioner prompt, generating a practitioner first query for the patient based on the received patient data and the practitioner prompt, wherein the query is generated by a dental diagnosis module (DAIM) and generating at least one of a practitioner second query or a dental diagnosis for the patient based on the response to the practitioner first dental query to enhance practitioner-patient communication.

Further yet in an embodiment of the invention, the system described is a computer program designed to facilitate doctor-patient communication in the context of dental care. The system includes a dental diagnosis module (DAIM), a processor, and a memory element coupled to the processor. The program is executable by the processor and is designed to operate over a network. The program is capable of receiving patient data and a practitioner prompt, which it uses to generate a practitioner first query for the patient. The practitioner first query is generated by training the DAIM using a combination of supervised fine-tuning and reinforcement learning techniques. The system can use a conversational interface, a chatbot or an AI-assistant to communicate with the patient and generate the practitioner first query. Based on the patient's response to the first query, the DAIM can generate at least one of a practitioner second query or a dental diagnosis to enhance practitioner-patient communication. Overall, the system described in the invention aims to improve the quality of doctor-patient communication in the context of dental care by using machine learning techniques to generate personalized queries and diagnoses that are tailored to the patient's specific needs and preferences.

Further yet in an embodiment of the invention, a method to generate a personalized medical summary from practitioner-patient communication comprising capturing a conversation between the practitioner and the patient, transcribing the conversation between the practitioner and the patient and generating the personalized medical summary for the patient based on the transcribed conversation, wherein the personalized medical summary is generated by a dental diagnosis module (DAIM). Additionally, an embodiment of the invention further comprising a recording device for capturing practitioner-patient communications wherein the recording device is at least one of voice recorders, smart phones, smart & digital devices, microphones, cameras, audio or video recorder, and digital transcription devices. The above-mentioned method has the potential to significantly improve the quality of medical care. By improving the accuracy and completeness of medical records, increasing efficiency, improving communication, and reducing risk, this method can help to ensure that patients receive the best possible care.

### BRIEF DESCRIPTION OF THE DRAWINGS

FIG. 1A illustrates in a block diagram, an automated parsing pipeline system for anatomical localization and condition classification, according to an embodiment.

FIG. 1B illustrates in a block diagram, an automated parsing pipeline system for anatomical localization and condition classification, according to another embodiment.

FIG. 2A illustrates in a block diagram, an automated parsing pipeline system for anatomical localization and condition classification according to yet another embodiment.

FIG. 2B illustrates in a block diagram, a processor system according to an embodiment.

FIG. 3A. illustrates in a flow diagram, an automated parsing pipeline method for anatomical localization and condition classification, according to an embodiment.

FIG. 3B illustrates in a flow diagram, an automated parsing pipeline method for anatomical localization and condition classification, according to another embodiment.

FIG. 4 illustrates in a block diagram, the automated parsing pipeline architecture according to an embodiment.

FIG. 5 illustrates in a screenshot, an example of ground truth and predicted masks in an embodiment of the present invention.

FIG. 6A illustrates in a screenshot, the extraction of anatomical structure by the localization model of the system in an embodiment of the present invention.

FIG. 6B illustrates in a screenshot, the extraction of anatomical structure by the localization model of the system in an embodiment of the present invention.

FIG. 6C illustrates in a screenshot, the extraction of anatomical structure by the localization model of the system in an embodiment of the present invention.

FIG. 7 illustrates in a graph, receiver operating characteristic (ROC) curve of a predicted tooth condition in an embodiment of the present invention.

FIG. 8 illustrates a method flow diagram of exemplary steps in the construction of an EoI-focused panorama in accordance with an aspect of the invention.

FIG. 9 illustrates in a screen-shot, an exemplary constructed panorama in accordance with an aspect of the invention.

FIG. 10 depicts a system block diagram of the EoI-focused panorama construction in accordance with an aspect of the invention.

FIG. 11 illustrates in a block diagram, an automated system for localizing and enumerating dental images according to an embodiment.

FIG. 12 illustrates in a block diagram, an automated system for localizing, enumerating, and diagnosing tooth conditions from dental images according to an embodiment.

FIG. 13 illustrates in a method flow diagram, the steps for localizing, enumerating, and diagnosing tooth conditions from dental images according to an embodiment.

FIG. 14 illustrates in a method flow diagram, the steps for localizing, enumerating, and diagnosing tooth conditions from dental images according to an embodiment.

FIG. 15 illustrates in a method flow diagram, the steps for localizing, enumerating, and diagnosing tooth conditions from dental images according to an embodiment.

FIG. 16 illustrates in a screenshot, an example of a sorted image according to an embodiment of the present invention.

FIG. 17 illustrates in a screenshot, an example of a localized and annotated tooth/teeth in an embodiment of the present invention.

FIG. 18 illustrates in a screenshot, an example of a localized, annotated, and diagnosed tooth/teeth in an embodiment of the present invention.

FIG. 19 illustrates in a screenshot, an example of a localized, annotated, and diagnosed tooth/teeth in an embodiment of the present invention.

FIG. 20 illustrates in a block diagram, an automated system for quick-glance layering localized and enumerated dental images according to an embodiment.

FIG. 21 illustrates in a block diagram, an automated system for quick-glance layering localized and enumerated dental images according to an embodiment.

FIG. 22 illustrates in a block diagram, an automated system for quick-glance layering localized and enumerated dental images according to an embodiment.

FIG. 23a illustrates an exemplary frame of an image-based layer according to an embodiment.

FIG. 23b illustrates an exemplary frame of an image based layer according to an embodiment.

FIG. 24 illustrates in a block diagram, an automated system for quick-glance layering according to an embodiment.

FIG. 25 illustrates an exemplary frame of an informational layer according to an embodiment.

FIG. 26 illustrates in a block diagram, an automated system for quick-glance according to an embodiment.

FIG. 27 illustrates an exemplary frame of an infographic layer according to an embodiment.

FIG. 28 illustrates an exemplary frame of interactive layers according to an embodiment.

FIG. 29 illustrates an exemplary frame of interactive layers according to an embodiment.

FIG. 30 illustrates an exemplary frame of interactive layers according to an embodiment

FIG. 31 illustrates in a method flow diagram, the steps for quick-glance layering according to an embodiment.

FIG. 32A illustrates a network diagram, according to an embodiment of the invention.

FIG. 32B illustrates a network diagram, according to an embodiment of the invention.

FIG. 33 illustrates a block diagram, according to an embodiment of the invention.

FIG. 34 illustrates a block diagram, according to an embodiment of the invention.

FIG. 35 illustrates a method flow diagram, according to an embodiment of the invention.

FIG. 36 illustrates a method flow diagram, according to an embodiment of the invention.

FIG. 37 illustrates a block diagram, according to an embodiment of the invention.

### DETAILED DESCRIPTION

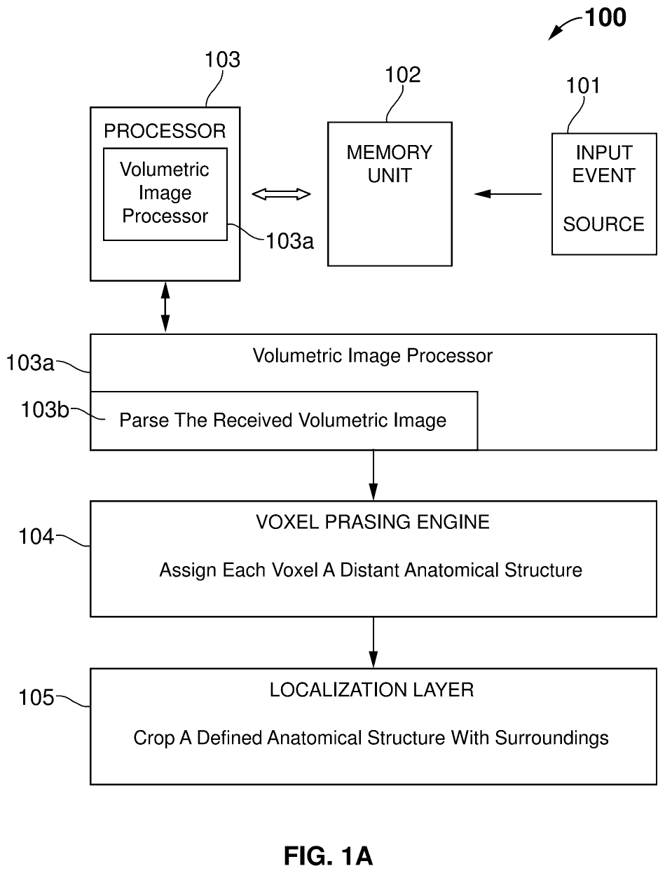

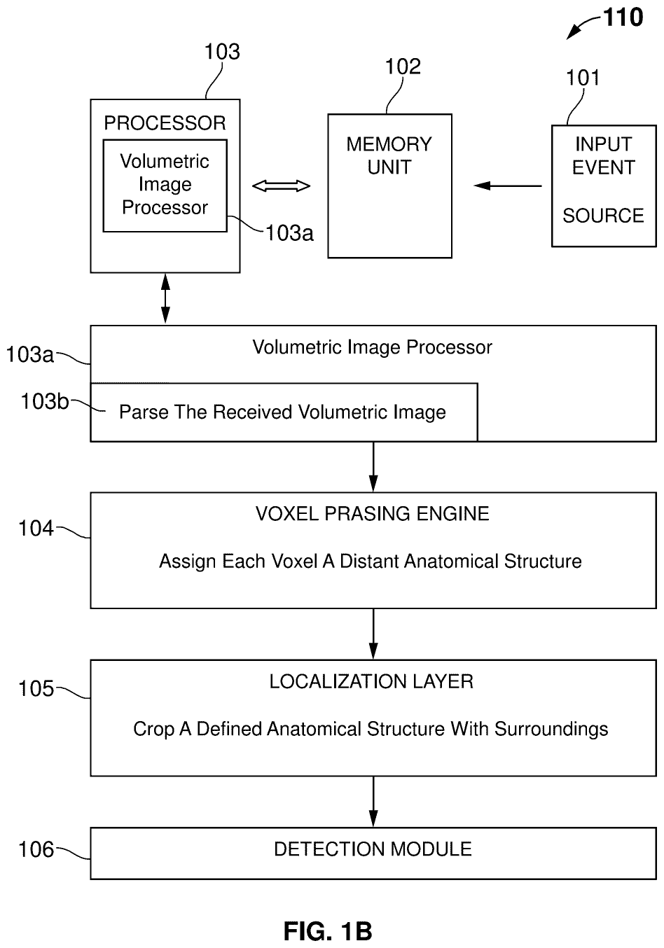

Specific embodiments of the invention will now be described in detail with reference to the accompanying FIGS. 1A-40. In the following detailed description of embodiments of the invention, numerous specific details are set forth in order to provide a more thorough understanding of the invention. In other instances, well-known features have not been described in detail to avoid obscuring the invention. Embodiments disclosed include an automated parsing pipeline system and method for anatomical localization and condition classification.

FIG. 1A illustrates a block diagram 100 of the system comprising an input event source 101, a memory unit 102 in communication with the input event source 101, a processor 103 in communication with the memory unit 102, a volumetric image processor 103a in communication with the processor 103, a voxel parsing engine 104 in communication with the volumetric image processor 103a and a localizing layer 105 in communication with the voxel parsing engine 104. In an embodiment, the memory unit 102 is a non-transitory storage element storing encoded information. The encoded instructions when implemented by the processor 103, configure the automated pipeline system to localize an anatomical structure and classify the condition of the localized anatomical structure.

In one embodiment, an input data is provided via the input event source 101. In one embodiment, the input data is a volumetric image data and the input event source 101 is a radio-image gathering source. In one embodiment, the input data is a 2-Dimensional (2D) image data. In another embodiment, the input data is a 3-Dimensional (3D) image data. The volumetric image processor 103a is configured to receive the volumetric image data from the radio-image gathering source. Initially, the volumetric image data is pre-processed, which involves conversion of 3-D pixel array into an array of Hounsfield Unit (HU) radio intensity measurements.

The processor 103 is further configured to parse at least one received image or volumetric image data 103b (i/v.i) into at least a single image frame field of view by the volumetric image processor. The processor 103 is further configured to localize anatomical structures residing in the single image frame field of view by assigning at least one of each a pixel or voxel (p/v) a distinct anatomical structure by the voxel parsing engine 104. In one embodiment, the single image frame field of view is pre-processed for localization, which involves rescaling using linear interpolation. The pre-processing involves use of any one of a normalization schemes to account for variations in image value intensity depending on at least one of an input or output of an image or volumetric image (i/v.i). In one embodiment, localization is achieved using any one of fully convolutional network or plain classification convolutional neural network (FCN/CNN), such as a V-Net-based fully convolutional neural network. In one embodiment, the V-Net is a 3D generalization of UNet.

The processor 103 is further configured to select all p/v belonging to the localized anatomical structure by finding a minimal bounding rectangle around the p/v and the surrounding region for cropping as a defined anatomical structure by the localization layer. The bounding rectangle extends equally in all directions to capture the tooth and surrounding context. In one embodiment, the bounding rectangle may extend 8-15 mm in all directions to capture the tooth and surrounding context.

FIG. 1B illustrates in a block diagram 110, an automated parsing pipeline system for anatomical localization and condition classification, according to another embodiment. The automated parsing pipeline system further comprises a detection module 106. The processor 103 is configured to detect or classify the conditions for each defined anatomical structure within the cropped image by a detection module or classification layer 106. In one embodiment, the classification is achieved using any one of a FCN/CNN, such as a DenseNet 3-D convolutional neural network.

In one embodiment, the localization layer 105 includes 33 class semantic segmentation in 3D. In one embodiment, the system is configured to classify each p/v as one of 32 teeth or background and resulting segmentation assigns each p/v to one of 33 classes. In another embodiment, the system is configured to classify each p/v as either tooth or other anatomical structure of interest. In case of localizing only teeth, the classification includes, but not limited to, 2 classes. Then individual instances of every class (teeth) could be split, e.g. by separately predicting a boundary between them. In some embodiments, the anatomical structure being localized, includes, but not limited to, teeth, upper and lower jaw bone, sinuses, lower jaw canal and joint.

In one embodiment, the system utilizes fully-convolutional network. In another embodiment, the system works on downscaled images (typically from 0.1-0.2 mm i/v.i resolution to 1.0 mm resolution) and grayscale (1-channel) image (say, 1×100×100×100-dimensional tensor). In yet another embodiment, the system outputs 33-channel image (say, 33×100×100×100-dimensional tensor) that is interpreted as a probability distribution for non-tooth vs. each of 32 possible (for adult human) teeth, for every p/v.

In an alternative embodiment, the system provides 2-class segmentation, which includes labelling or classification, if the localization comprises tooth or not. The system additionally outputs assignment of each tooth p/v to a separate “tooth instance”.

In one embodiment, the system comprises FCN/CNN (such as VNet) predicting multiple “energy levels”, which are later used to find boundaries. In another embodiment, a recurrent neural network could be used for step by step prediction of tooth, and keep track of the teeth that were outputted a step before. In yet another embodiment, Mask-RCNN generalized to 3D could be used by the system. In yet another embodiment, the system could take multiple crops from 3D image in original resolution, perform instance segmentation, and then join crops to form mask for all original image. In another embodiment, the system could apply either segmentation or object detection in 2D, to segment axial slices. This would allow to process images in original resolution (albeit in 2D instead of 3D) and then infer 3D shape from 2D segmentation.

In one embodiment, the system could be implemented utilizing descriptor learning in the multitask learning framework i.e., a single network learning to output predictions for multiple dental conditions. This could be achieved by balancing loss between tasks to make sure every class of every task have approximately same impact on the learning. The loss is balanced by maintaining a running average gradient that network receives from every class*task and normalizing it. Alternatively, descriptor learning could be achieved by teaching network on batches consisting data about a single condition (task) and sample examples into these batches in such a way that all classes will have same number of examples in batch (which is generally not possible in multitask setup). Further, standard data augmentation could be applied to 3D tooth images to perform scale, crop, rotation, vertical flips. Then, combining all augmentations and final image resize to target dimensions in a single affine transform and apply all at once.

Advantageously, in some embodiments, to accumulate positive cases faster, a weak model could be trained and ran for all of the unlabeled data. From resulting predictions, teeth model that yield high scores on some rare pathology of interest are selected. Then the teeth are sent to be labelled and added to the dataset (both positive and negative labels). This allows to quickly and cost-efficiently build up a more balanced dataset for rare pathologies.

In some embodiments, the system could use coarse segmentation mask from localizer as an input instead of tooth image. In some embodiments, the descriptor could be trained to output fine segmentation mask from some of the intermediate layers. In some embodiments, the descriptor could be trained to predict tooth number.

As an alternative to multitask learning approach, “one network per condition” could be employed, i.e. models for different conditions are completely separate models that share no parameters. Another alternative is to have a small shared base network and use separate subnetworks connected to this base network, responsible for specific conditions/diagnoses.

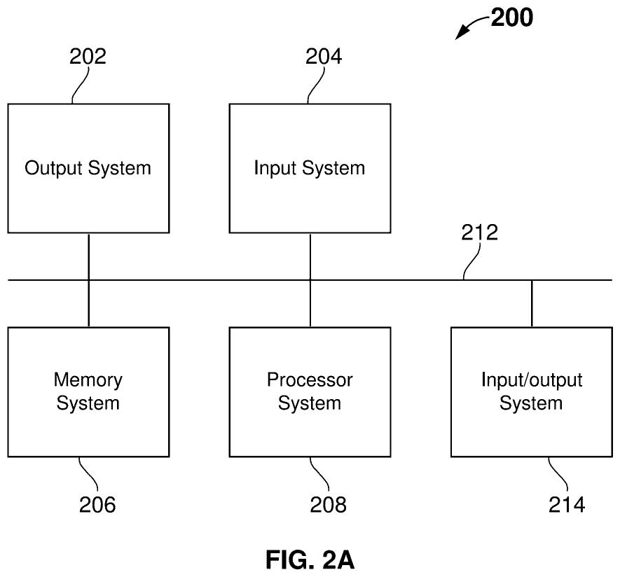

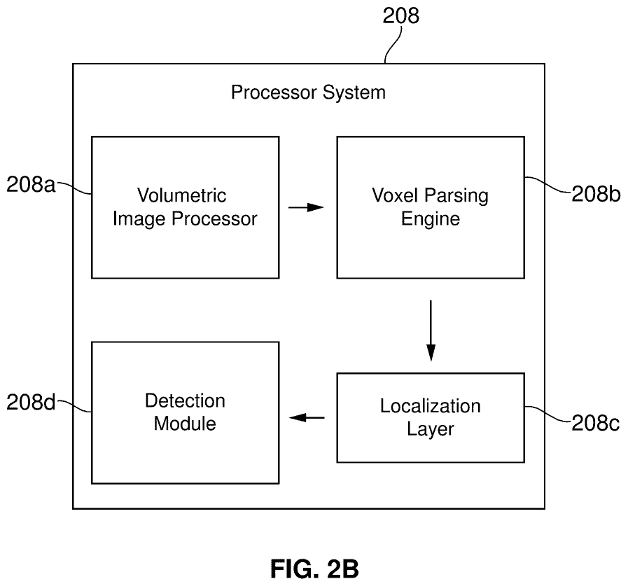

FIG. 2A illustrates in a block diagram 200, an automated parsing pipeline system for anatomical localization and condition classification according to yet another embodiment. In an embodiment, the system comprises an input system 204, an output system 202, a memory system or unit 206, a processor system 208, an input/output system 214 and an interface 212. Referring to FIG. 2B, the processor system 208 comprises a volumetric image processor 208a, a voxel parsing engine 208b in communication with the volumetric image processor 208a, a localization layer 208c in communication with the voxel parsing engine 208 and a detection module 208d in communication with the localization module 208c. The processor 208 is configured to receive at least one i/v.i via an input system 202. At least one received i/v.i, which may be comprised of a 2-D or 3-D image. The pixel array is pre-processed to convert into an array of Hounsfield Unit (HU) radio intensity measurements. Then, the processor 208 is configured to parse the received i/v.i into at least a single image frame field of view by the said volumetric image processor 208a.

The anatomical structures residing in the at least single field of view is localized by assigning each p/v a distinct anatomical structure by the voxel parsing engine 208b. The processor 208 is configured to select all p/v belonging to the localized anatomical structure by finding a minimal bounding rectangle around the p/v and the surrounding region for cropping as a defined anatomical structure by the localization layer 208c. Then, the conditions for each defined anatomical structure within the cropped image is classified by a detection module or classification layer 208d.

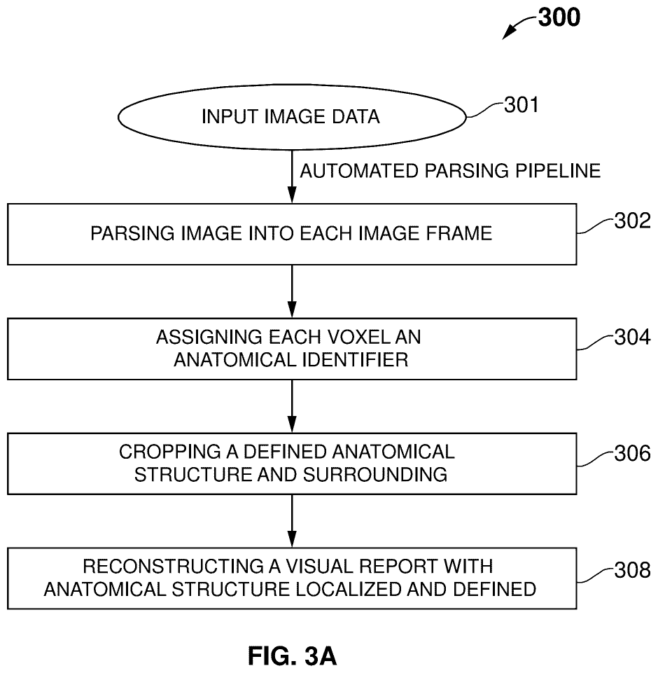

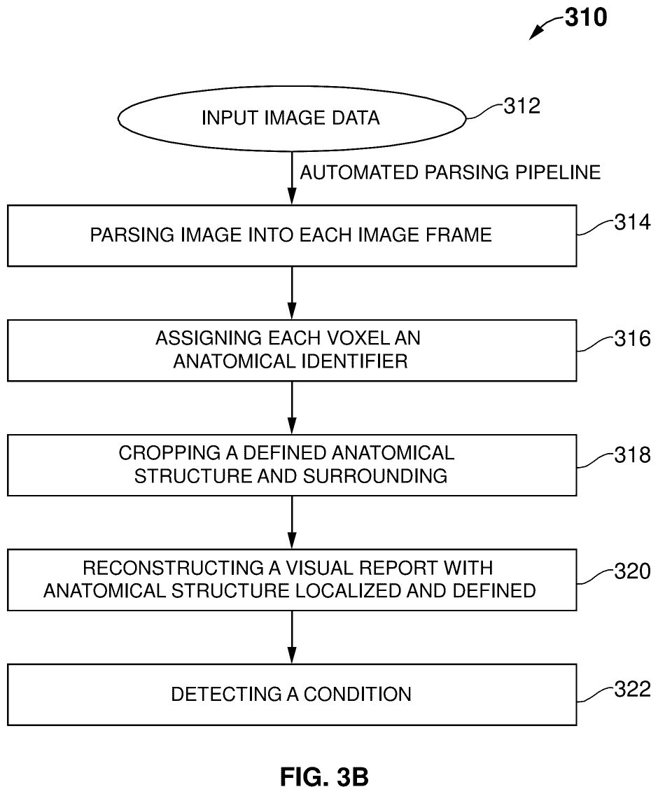

FIG. 3A illustrates in a flow diagram 300, an automated parsing pipeline method for anatomical localization and condition classification, according to an embodiment. At step 301, an input image data is received. In one embodiment, the image data is a i/v.i. At step 302, the received i/v.i is parsed into at least a single image frame field of view. The parsed i/v.i is pre-processed by controlling image intensity value.

At step 304, a tooth or anatomical structure inside the pre-processed and parsed i/v.i is localized and identified by tooth number. At step 306, the identified tooth and surrounding context within the localized i/v.i are extracted. At step 308, a visual report is reconstructed with localized and defined anatomical structure. In some embodiments, the visual reports include, but not limited to, an endodontic report (with focus on tooth's root/canal system and its treatment state), an implantation report (with focus on the area where the tooth is missing), and a dystopic tooth report for tooth extraction (with focus on the area of dystopic/impacted teeth).

FIG. 3B illustrates in a flow diagram 310, an automated parsing pipeline method for anatomical localization and condition classification, according to another embodiment. At step 312, at least one i/v.i is received from a radio-image gathering source by a volumetric image processor.

At step 314, the received i/v.i is parsed into at least a single image frame field of view by the volumetric image processor. At least single image frame field of view is pre-processed by controlling image intensity value by the volumetric image processor. At step 316, an anatomical structure residing in the at least single pre-processed field of view is localized by assigning each p/v a distinct anatomical structure ID by the voxel parsing engine. At step 318, all p/v belonging to the localized anatomical structure is selected by finding a minimal bounding rectangle around the p/v and the surrounding region for cropping as a defined anatomical structure by the localization layer. At step 320, a visual report is reconstructed with defined and localized anatomical structure. At step 322, conditions for each defined anatomical structure is classified within the cropped image by the classification layer.

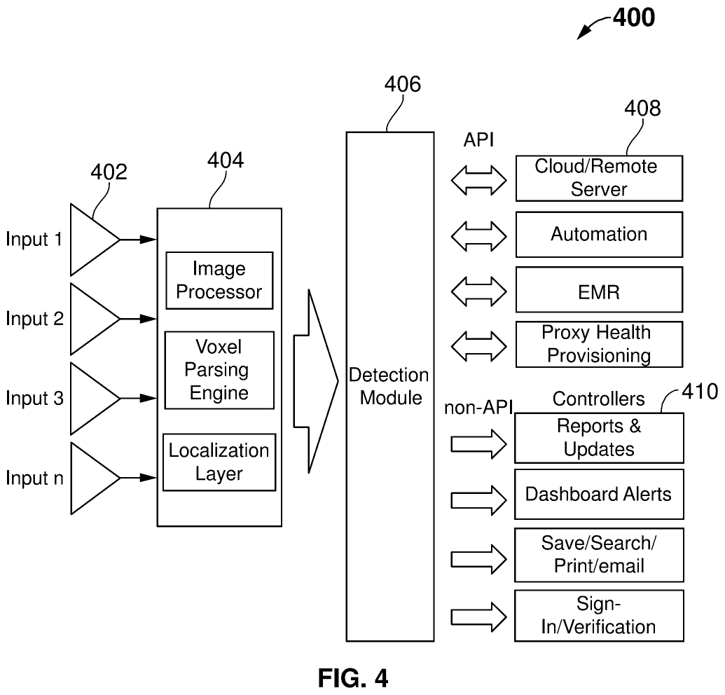

FIG. 4 illustrates in a block diagram 400, the automated parsing pipeline architecture according to an embodiment. According to an embodiment, the system is configured to receive input image data from a plurality of capturing devices, or input event sources 402. A processor 404 including an image processor, a voxel parsing engine and a localization layer. The image processor is configured to parse image into each image frame and preprocess the parsed image. The voxel parsing engine is configured to configured to localize an anatomical structure residing in the at least single pre-processed field of view by assigning each p/v a distinct anatomical structure ID. The localization layer is configured to select all p/v belonging to the localized anatomical structure by finding a minimal bounding rectangle around the p/v and the surrounding region for cropping as a defined anatomical structure. The detection module 406 is configured to detect the condition of the defined anatomical structure. The detected condition could be sent to the cloud/remote server, for automation, to EMR and to proxy health provisioning 408. In another embodiment, detected condition could be sent to controllers 410. The controllers 410 includes reports and updates, dashboard alerts, export option or store option to save, search, print or email and sign-in/verification unit.

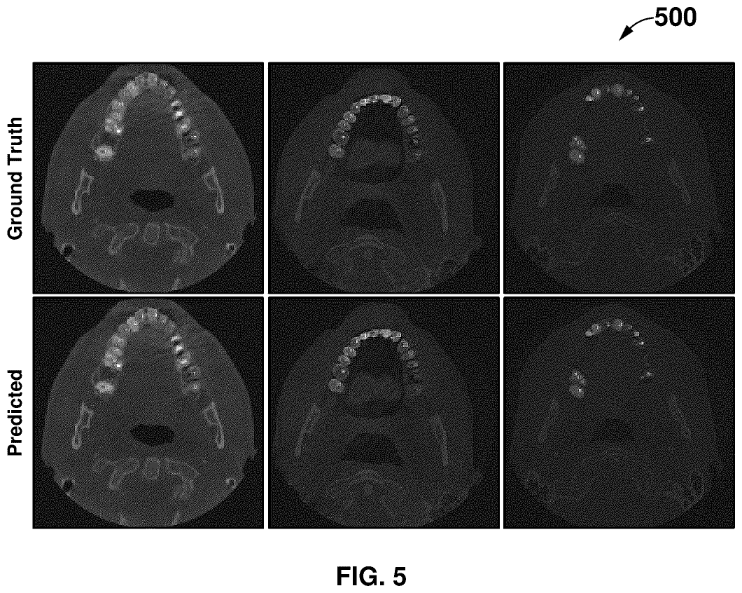

Referring to FIG. 5, an example screenshot 500 of tooth localization done by the present system, is illustrated. This figure shows examples of teeth segmentation at axial slices of 3D tensor.

Problem: Formulating the problem of tooth localization as a 33-class semantic segmentation. Therefore, each of the 32 teeth and the background are interpreted as separate classes.

Model: A V-Net-based fully convolutional network is used. V-Net is a 6-level deep, with widths of 32; 64; 128; 256; 512; and 1024. The final layer has an output width of 33, interpreted as a SoftMax distribution over each voxel, assigning it to either the background or one of 32 teeth. Each block contains 3*3*3 convolutions with padding of 1 and stride of 1, followed by ReLU non-linear activations and a dropout with 0:1 rate. Instance normalization before each convolution is used. Batch normalization was not suitable in this case, as long as there is only one example in batch (GPU memory limits); therefore, batch statistics are not determined.

Different architecture modifications were tried during the research stage. For example, an architecture with 64; 64; 128; 128; 256; 256 units per layer leads to the vanishing gradient flow and, thus, no training. On the other hand, reducing architecture layers to the first three (three down and three up) gives a comparable result to the proposed model, though the final loss remains higher.

Loss function: Let R be the ground truth segmentation with voxel values ri (0 or 1 for each class), and P the predicted probabilistic map for each class with voxel values pi. As a loss function we use soft negative multi-class Jaccard similarity, that can be defined as:

$$Jaccard Multi class Loss = 1 - \frac{1}{N} {\sum }_{i = 0}^{N} \frac{{p}_{i} {r}_{i} + ∈}{{p}_{i} + {r}_{i} - {p}_{i} {r}_{i} + {∈}^{′}}$$

where N is the number of classes, which in our case is 32, and E is a loss function stability coefficient that helps to avoid a numerical issue of dividing by zero. Then the model is trained to convergence using an Adam optimizer with learning rate of 1e−4 and weight decay 1e−8. A batch size of 1 is used due to the large memory requirements of using volumetric data and models. The training is stopped after 200 epochs and the latest checkpoint is used (validation loss does not increase after reaching the convergence plateau).

Results: The localization model is able to achieve a loss value of 0:28 on a test set. The background class loss is 0:0027, which means the model is a capable 2-way “tooth/not a tooth” segmentor. The localization intersection over union (IoU) between the tooth's ground truth volumetric bounding box and the model-predicted bounding box is also defined. In the case where a tooth is missing from ground truth and the model predicted any positive p/v (i.e. the ground truth bounding box is not defined), localization IoU is set to 0. In the case where a tooth is missing from ground truth and the model did not predict any positive p/v for it, localization IoU is set to 1. For a human-interpretable metric, tooth localization accuracy which is a percent of teeth is used that have a localization IoU greater than 0:3 by definition. The relatively low threshold value of 0:3 was decided from the manual observation that even low localization IoU values are enough to approximately localize teeth for the downstream processing. The localization model achieved a value of 0:963 IoU metric on the test set, which, on average, equates to the incorrect localization of 1 of 32 teeth.

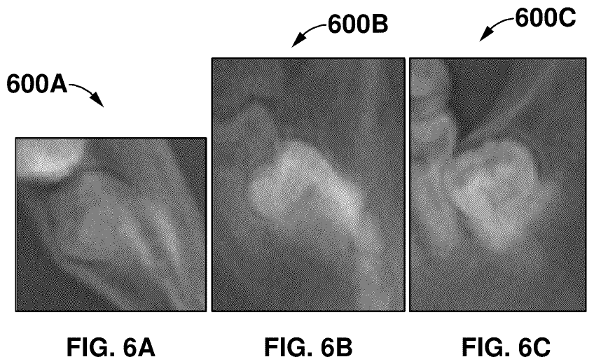

Referring to FIGS. 6A-6C, an example screenshot (600A, 600B, 600B) of tooth sub-volume extraction done by the present system, illustrated.

In order to focus the downstream classification model on describing a specific tooth of interest, the tooth and its surroundings is extracted from the original study as a rectangular volumetric region, centered on the tooth. In order to get the coordinates of the tooth, the upstream segmentation mask is used. The predicted volumetric binary mask of each tooth is preprocessed by applying erosion, dilation, and then selecting the largest connected component. A minimum bounding rectangle is found around the predicted volumetric mask. Then, the bounding box is extended by 15 mm vertically and 8 mm horizontally (equally in all directions) to capture the tooth and surrounding region (tooth context) and to correct possibly weak localizer performance. In other embodiments, the minimum bounding box may be any length in either direction to optimally capture tooth context. Finally, a corresponding sub-volume is extracted from the original clipped image, rescale it to 643 and pass it on to the classifier. An example of a sub-volume bounding box is presented in FIGS. 6A-6C.

| | Artificial | Filling | | Impacted | | |
|---|---|---|---|---|---|---|
| | crowns | canals | Filling | tooth | Implant | Missing |
| ROC AUC | 0.941 | 0.95 | 0.892 | 0.931 | 0.979 | 0.946 |
| Condition | 0.092 | 0.129 | 0.215 | 0.018 | 0.015 | 0.145 |
| frequency | | | | | | |

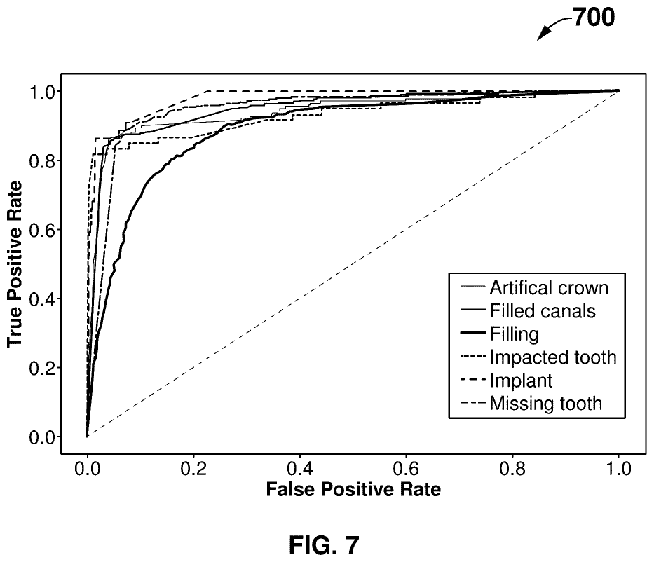

Referring to FIG. 7, a receiver operating characteristic (ROC) curve 700 of a predicted tooth condition is illustrated.

Model: The classification model has a DenseNet architecture. The only difference between the original and implementation of DenseNet by the present invention is a replacement of the 2D convolution layers with 3D ones. 4 dense blocks of 6 layers is used, with a growth rate of 48, and a compression factor of 0:5. After passing the 643 input through 4 dense blocks followed by down-sampling transitions, the resulting feature map is 548×2×2×2. This feature map is flattened and passed through a final linear layer that outputs 6 logits each for a type of abnormality.

Loss function: Since tooth conditions are not mutually exclusive, binary cross entropy is used as a loss. To handle class imbalance, weight each condition loss inversely proportional to its frequency (positive rate) in the training set. Suppose that Fi is the frequency of condition i, pi is its predicted probability (sigmoid on output of network) and ti is ground truth. Then: Li=(1=Fi). ti·log pi+Fi·(1−ti)·log(1−pi) is the loss function for condition i. The final example loss is taken as an average of the 6 condition losses.

Results: The classification model achieved average area under the receiver operating characteristic curve (ROC AUC) of 0:94 across the 6 conditions. Per-condition scores are presented in above table. Receiver operating characteristic (ROC) curves 700 of the 6 predicted conditions are illustrated in FIG. 7.

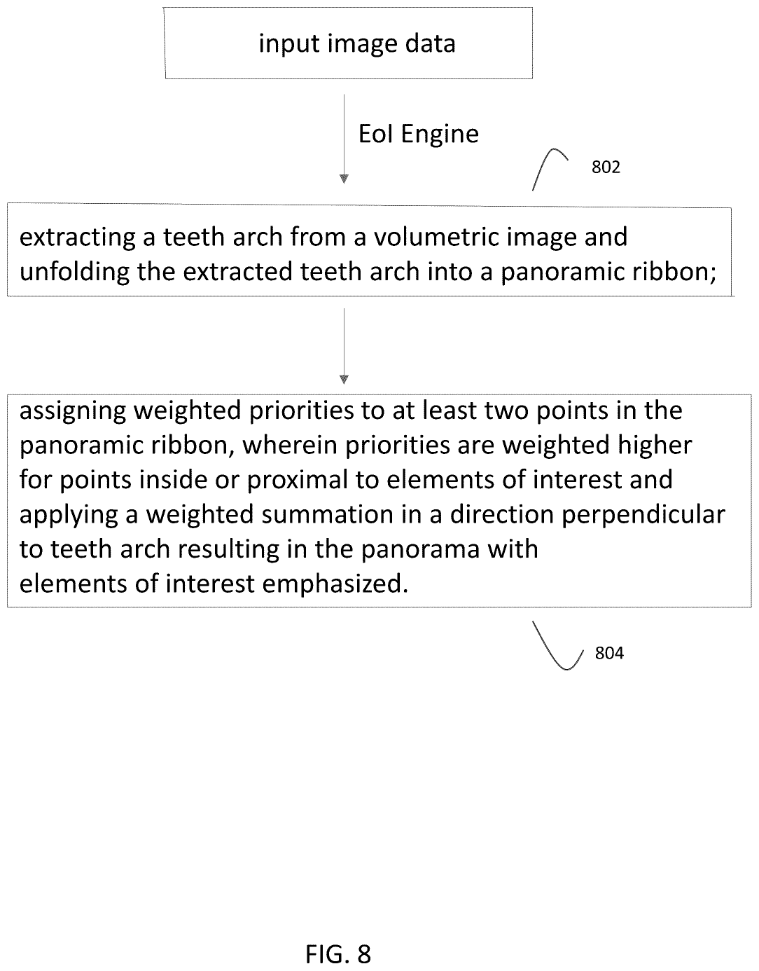

FIG. 8 illustrates a method flow diagram of exemplary steps in the construction of an EoI-focused panorama in accordance with an aspect of the invention. It is to be understood that references to anatomical structures may also assume image or image data corresponding to the structure. For instance, extracting a teeth arch translates to extracting the portion of the image wherein the teeth arch resides, and not the literal anatomical structure. FIG. 8 generally illustrates a method of steps for constructing a 2D panoramic image from a 3D CBCT study. More specifically, in one embodiment, disclosed is a method for constructing a panorama of teeth arch with elements of interest emphasized. In a preferred embodiment, the method comprises the step of: (1) extracting a teeth arch from a volumetric image and unfolding the extracted teeth arch into a panoramic ribbon 802; and step (2) assigning weighted priorities to at least two points in the panoramic ribbon, wherein priorities are weighted higher for points inside or proximal to elements of interest (EoI) and applying a weighted summation in a direction perpendicular to teeth arch resulting in the panorama with EoI emphasized 804.

The construction of the EoI-focused panorama of an oral complex (or more specifically, a teeth arch) is generally achieved in two steps according to one embodiment: The first step concludes in voxel coordinates operations, such as extracting teeth arch and unfolding a study image into 3D panoramic ribbon (curved sub-volume that passes along the teeth arch). Teeth arch is extracted using segmentations of teeth and anatomy, mandible and mandibular canals in particular. Anatomy segmentation may also required to maintain stable extraction in case of missing teeth and to ensure that TMJs and canals are visualized in panorama. Then, construct a transformation grid that allows the arch to unfold in straight line, resulting in a panoramic ribbon. In one embodiment, the transformation grid is a calculation of the coordinates according to the coordinates of the original volumetric image. Once unfolded, in one embodiment, the resulting image is virtually tilted in sagittal plane for frontal teeth apexes to take the most perceptible position. Tilting of the ribbon to maximize perceptibility of a frontal teeth apex is done by calculating the angles of frontal section tilt for both sections, applying the transformations according to the calculated tilt during the process of calculating of the coordinates of a transformation grid, so the panoramic ribbon is tilted virtually in non-distorting manner. Alternatively, the unfolding of the arch in a straight line for generating the ribbon does not require applying a transformation grid. In other embodiments, the unfolding of the arch into the ribbon does not subsequently tilt to maximize frontal teeth apex perceptibility.

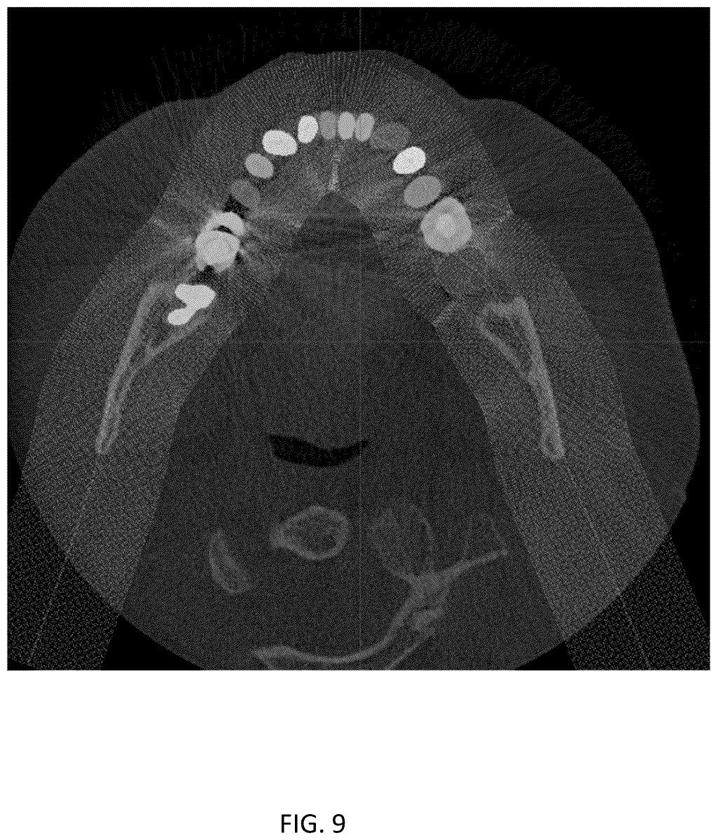

During the second step we assign priorities to each point in panoramic ribbon. Priorities are defined as high in points that are inside or close to regions of interest (such as teeth, bone, and mandibular canals) and as low in points far from them. Final panoramic image is obtained by weighted summation in the direction perpendicular to teeth arch, where weights are priorities. This results in a panorama where elements of interest are emphasized in non-distorting manner, as illustrated in FIG. 9 (screen-shot of an exemplary constructed EoI-focused panorama in accordance with an aspect of the invention). In general, two types of panoramas may be constructed using the above mentioned method: (1) a general panorama; and (2) a split panorama. A general panorama includes RoI for all the teeth present, a whole mandible with TMJs, and a lower part of maxilla with sinuses cropped to the middle. A split panorama is constructed for both jaws separately and is focused only on teeth—as shown in FIG. 9. Alternatively, a method for generating a split or general panorama may be done with the following steps, beginning with (1) combining teeth and mandible segmentations; (2) building an axial projection of segmentation combination; (3) building a skeleton of the axial projection; (4) dividing the skeleton into all possible paths; (5) taking a longest path; (6) ensuring that this path is long enough to cover TMJs, if they are present; (7) smoothing the path; and then (8) returning it as the teeth arch.

While not illustrated in FIG. 8, one embodiment of the method flow may entail: As a first step, receiving an i/v.i (image/volumetric image) further parsed into at least a single image frame field of view by the volumetric image processor. Optionally, the at least single image frame field of view may be pre-processed by controlling image intensity value by the volumetric image processor. As a next step, an anatomical structure residing in the at least single field of view is localized by assigning each p/v (pixel/voxel) a distinct anatomical structure ID by the voxel parsing engine. Next, all p/v belonging to the localized anatomical structure is selected by finding a minimal bounding rectangle around the p/v and the surrounding region for cropping as a defined anatomical structure by the localization layer. Optionally, a visual report may be reconstructed with defined and localized anatomical structure. Also optionally, conditions for each defined anatomical structure may be classified within the cropped image by the classification layer. Next, extracting a teeth arch from the localized image generated and unfolding the extracted teeth arch into a panoramic ribbon; and finally, assigning weighted priorities to at least two points in the panoramic ribbon, wherein priorities are weighted higher for points inside or proximal to elements of interest (EoI) and applying a weighted summation in a direction perpendicular to teeth arch resulting in the panorama with EoI emphasized.

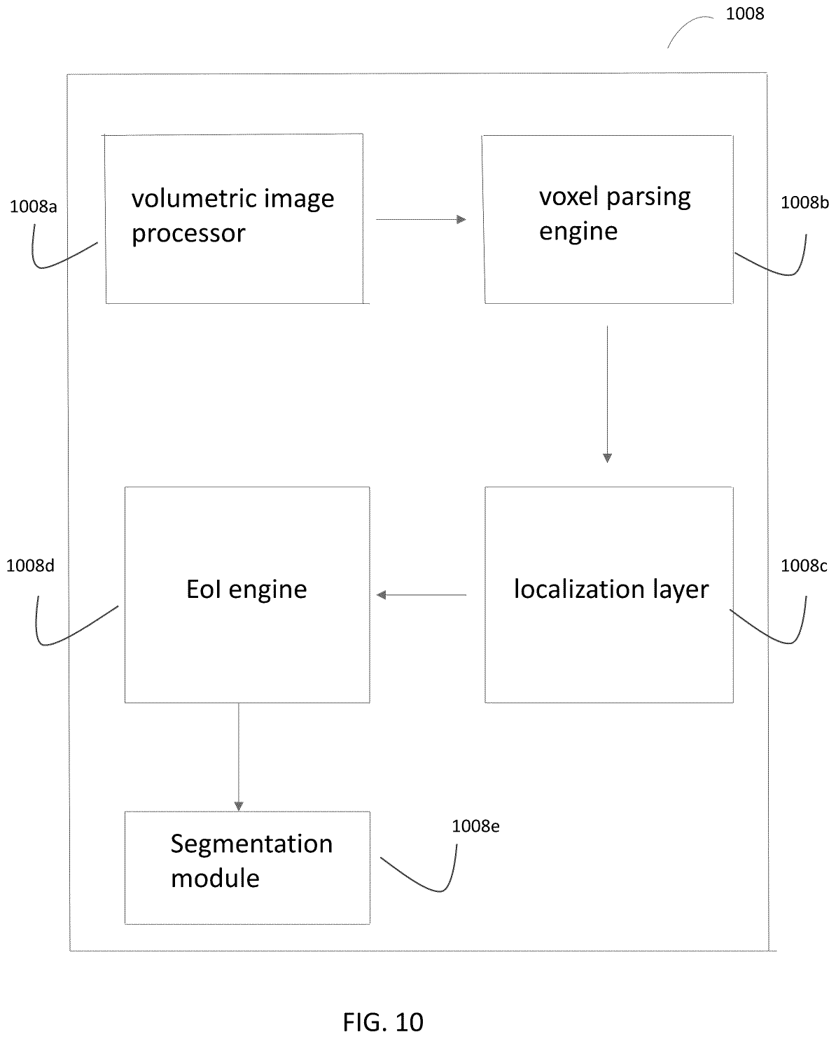

Now in reference to FIG. 10 depicting a system block diagram of the EoI-focused panorama construction in accordance with an aspect of the invention. A system for constructing an Elements of Interest (EoI)-focused panorama may comprise: a processor 1008; a non-transitory storage element coupled to the processor 1008; encoded instructions stored in the non-transitory storage element, wherein the encoded instructions when implemented by the processor 1008, configure the system to: extract a teeth arch from a volumetric image; unfold the extraction into a panoramic ribbon; assign weighted priorities to each point in the ribbon, wherein priorities are weighted higher for points inside or proximal to an EoI; and apply a weighted summation in a direction perpendicular to teeth arch, resulting in the panorama with the EoI emphasized.

As shown in FIG. 10, the system may comprise: a volumetric image processor 1008a, a voxel parsing engine 1008b, a localization layer 1008c, an EoI engine 1008d, an instance segmentation module 1008e, wherein the system, and more particularly, the EoI engine 1008d may be configured to: extract a teeth arch from a volumetric image; form a study image from the extract; unfold the study image into a panoramic ribbon; tilt the ribbon for maximal frontal teeth exposure; assign weighted priorities to at least two points in the ribbon, wherein priorities are weighted higher for points inside or proximal to EoI; apply a weighted summation in a direction perpendicular to teeth arch resulting in the panorama with EoI emphasized; and apply the instance segmentation module 1008e to accurately provide a segmentation mask and numbering with EoI emphasized.

In one embodiment, the teeth arch is extracted using segmentations of at least one of a teeth or anatomy by the EoI engine 1008d. Alternatively, the teeth arch extraction is done by an algorithm that combines teeth and mandible segmentations, extracts the teeth arch landmarks in 2D plane, fits a pre-defined function in form of a default teeth arch to the extracted landmarks, and returns a fitted function as the teeth arch. In one embodiment, the panoramic ribbon is a curved sub-volume passing along the teeth arch, created from unfolding the image of the extracted teeth arch. Alternatively, the extension of the teeth arch from 2D plane to 3D is done by an algorithm that extracts vestibuloral slices from a curved sub-volume of teeth and anatomy segmentations passing along the 2D teeth arch; construct a vertical curve that approximates position and tilt of upper and lower teeth and anatomy on each axial level at each vestibuloral slice; combine the extracted vertical curves in a way that points of each curve on each axial level resulting in a 2D teeth arch specific for a given axial level; and return a vertical curve combination as a 3D teeth arch.

In an embodiment, once the 3D panoramic ribbon is generated, priorities are assigned to a plurality of points arbitrarily chosen on the panoramic ribbon. The arbitrarily chosen points are evenly spaced along the length of the panoramic ribbon. In other embodiments, the points may not be chosen arbitrarily, but rather, according to a pre-defined rule. The elements of interest may be at least one of a bone, tooth, teeth, or mandibular canals. wherein weights are assigned highest to points inside or most proximal to elements of interest with a pre-defined highest value.

While not shown in FIG. 10, in another embodiment, the system may be configured to receive input image data from a plurality of capturing devices, or input event sources. A processor, including or in addition to, an image processor, a voxel parsing engine, a localization layer, and an EoI Engine may be comprised within the system. An image processor may be configured to parse the captured image into each image frame and, optionally, preprocess the parsed image. A voxel parsing engine may be configured to localize an anatomical structure residing in the at least single field of view by assigning each p/v a distinct anatomical structure ID. The localization layer may be configured to select all p/v belonging to the localized anatomical structure by finding a minimal bounding rectangle around the p/v and the surrounding region for cropping as a defined anatomical structure. Optionally, a detection module may be configured to detect the condition of the defined anatomical structure. Also, optionally, the detected condition could be sent to the cloud/remote server, for automation, to EMR and to proxy health provisioning. In another embodiment, detected condition could be sent to controllers, for generating reports and updates, dashboard alerts, export option or store option to save, search, print or email and sign-in/verification unit. Once localized, the EoI engine may be configured to: extract a teeth arch from the localized image; optionally, form a study image from the extract; unfold the extracted teeth arch, or optionally, the study image into a panoramic ribbon; tilt the ribbon for maximal frontal teeth exposure; assign weighted priorities to at least two points in the ribbon, wherein priorities are weighted higher for points inside or proximal to EoI; apply a weighted summation in a direction perpendicular to teeth arch resulting in the panorama with EoI emphasized; and, optionally, apply the instance segmentation module to accurately provide a segmentation mask and numbering with EoI emphasized.

In one embodiment, a segmentation module 1008e (optionally, an instance segmentation module), operating over 2D panoramic image plane, provides accurate teeth segmentation masks and numbering to a corresponding 3D CBCT image. The initial step is to segment teeth instances on an automatically generated panoramic image. Using state-of-the-art 2D instance segmentation deep learning models (R-CNN detectors), 1) localize teeth in 2D bounding boxes; 2) assign a number to each detected tooth in accordance with the dental formula; and 3) provide accurate 2D masks to each detected tooth. To train 2D instance segmentation module, utilize the mixture of the annotated OPT and panoramic images generated from CBCT, obtained as an output of our automatic panoramic generator. With the assistance of the 3D panoramic ribbon, retrieve 3D bounding boxes, inferred from the 2D panoramic instances, defining correspondence between 2D and 3D bounding boxes coordinates. The 3D bounding boxes (as regions of interest) are further submitted to 3D segmentation module, to obtain accurate 3D teeth masks in the original CBCT fine scale.

In further clarification, using one of the module's output (3D-UNet or 3D-R-CNN), use teeth masks to automatically construct panoramic surface, which defines mathematically; independently project the 3D tooth masks of both modules on a panoramic surface which gives 2D tooth masks projections. In order do this, calculate normal vectors to the panoramic surface and project every pixel of a mask on this surface. In parallel with the previous step, apply the third, 2D R-CNN style instance segmentation model, to the generated panorama image to acquire 2D tooth masks and labels. For every 2D R-CNN mask, pick an instance either from 3D-UNet or 3D-R-CNN. This step is accomplished by calculating an Intersection over Union (IoU) for 2D masks and the projected 3D masks, selecting the best 3D projections for every 2D instance on the panoramic image detected by 2D R-CNN detector. Since the relations between the 3D projected masks and the 3D masks themselves are understood, picking a 3D projected mask infers picking the corresponding 3D mask.

In one embodiment, the segmentation module 1008e (optionally, semantic segmentation modules or 3D U-Nets) is configured to specifically segment localized pathologies, like caries and periapical lesions. We use these segmentation to 1) estimate volume of the lesions to track it size dynamic over time, 2) create very specific visualizations (slice that cuts right at the maximum volume of the lesion), 3) get a “second opinion” network for diagnostics. These networks could also be trained in multi-task fashion, where one network learns to segment multiple types of lesions. A classification module could also be attached to the outputs of any network layer(s) to produce probabilities for lesion or whole-image. In case of whole-image classifier, it could be used to add cheaper weakly labeled data (single whole-image label “image contains lesions”/“image does not contain lesions”) to costly segmentation labels (assigning lesion/background to each voxel). These networks typically operate on RoI defined by tooth sub-volume, like the Descriptor. However, non-dental diseases such as tumors and cysts in the jaws may also be diagnosed. In those cases, different RoI, like RoI of the upper/lower jaw, may be used.

Besides teeth, an anatomy of the skull may be segmented: Mandible and maxilla mandibular canal Sinuses, for instance. The combination of anatomy, along with teeth, may be exported as STL models to third party applications. The segmentation of mandibular canal to characterize a tooth's relation with it, e.g. “tooth is really close to canal”, may be performed. This is relevant for surgery planning and diagnostics. Furthermore, a mandible and maxilla segmentation may be performed to diagnose gum disease and to select RoI for additional processing (e.g. tooth segmentation). Even furthermore, sinuses may be segmented to select RoI for sinus diagnosis.

A root-canal system localization module may be used to accurately segment all roots, canals inside them and pulp chamber for each presented tooth. A 3D U-Net based CNN architecture may be used to solve a multiclass semantic segmentation problem. Precise roots and canals segmentation affords a diagnostician/practitioner to estimate canals length, their curvature and visualize the most informative tooth slices in any point, direction, and with any size and thickness. This module allows to see and understand the anatomy of dental roots and canals and forms the basis for planning an endodontic treatment.

Gum disease is a loss of bone around a tooth. Inflammation around teeth may cause bone to recede and expose a tooth's roots. Gum disease diagnosis is performed by measuring a bone loss from a cemento-enamel junction (CEJ, line on tooth's crown where enamel ends) to the beginning of bone envelope. Diagnosis is performed by segmenting 1) tooth's body, 2) enamel, 3) alveolar bone (tooth's bony envelope), and then algorithmically measuring what part of a tooth between apex and CEJ is covered by the bone.

These are two types of artefacts that may corrupt an image, rendering it unusable for diagnostic purposes. A model developed takes in 2D axial patches, centered on teeth, and predicts if the given CBCT is affected by an artefact, and also predicts the intensity of an artefact. If this probability is high, re-taking the image is recommended. In extreme cases, an intensity score may be assigned corresponding to the severity of the artefact.

A model developed finds locations of cephalometric landmarks on CBCT. Based on this, a calculation of cephalometric measurements may be performed, which are relations (distances and angles) between sets of landmarks. Based on measurements, a screening may be performed, which may signal if patient might have an aesthetic/functional orthodontic problem and would benefit from a consult with an orthodontic.

A report that serves as a guide for implantology planning for a specific area has also been developed. A report consists of a panorama and a group of vestibular slices. A panorama may be constructed using virtual rotation that aligns occlusal plane. A panorama serves as a topogram that shows the locations of every slice. Slices have a tilt that is intended to match the implant placement direction. Some of the slices are provided with measurements that are present only if there is a place for implant. The distance can be estimated 1) as a horizontal plate situated in the implant entrance area that is usually tilted in the implant placement direction, 2) from the center of the plate to the closest point of either mandibular canal or maxillary sinus, 3) as a vertical from the oral end of plate to the farthest point of mandible.

Also developed is an endodontic treatment planning report generated from an uploaded CBCT image and chosen area of interest on the dental formula. A series of slices are then obtained: axial, cross-sectional canal shape, periapical lesions, C-shaped root canal, and root furcation. The report consists of several modules: the panoramic image of upper and/or lower jaw (depends on the region of interest), root canal space, root canal system, root canal shape, function, and periapical lesions. Optionally, a root canal system anatomy may be assessed and an evaluation of possible endodontic pathology. Further optionally, a report may be generated, which can be stored or handed over to the patient.

A report may be generated that provides necessary visual information about a specific third molar using the knowledge of tooth location. A report consists of three slice sections that differ in slice orientation: vestibular, axial, and mesiodistal slices. Every slice section has a topogram image that shows slice locations. A mandibular canal segmentation may be performed to visualize its location on topograms to notify a surgeon about tooth-canal relation.

As a part of mandible/maxilla, TMJ parts: Condyle and temporal bone (this step is optional, landmarks could be detected) may be segmented. Then, several landmarks on condyle and temporal bone may be detected and distances between them measured. These measurements and their relations, e.g. asymmetry, may be a basis for TMJ diagnosis and referral for additional study via MRI. TMJ disorders can cause pain and are typically counter-indications for orthodontic treatment.

Similar to pathology localizers, a model has been developed that will segment pathologies on panoramic radiograph. It operates over the entire image or inside a tooth RoI (bounding box predicted by OPT localizer). Any type of 2D segmentation network can be used, e.g. UNet. After pathology segmentation is obtained, assign pathologies to tooth by selecting those that are inside predicted tooth mask or are immediately adjacent to it.

Superimposition of several CBCTs are important for assessing changes between two time points. This may be performed in one of the following ways:
1) General SI: predict ceph landmarks and orient one image onto another by minimizing the distance between individual ceph points on the image.
2) During superimposition, some landmarks can be ignored if they are significantly changed, i.e., we minimize distance between points EXCEPT N points that have maximum distances after algorithm has converged.
3) Tooth-related SI: predict some tooth landmarks, like apex/radix points, furcation points, crown “bumps” and “fissures”. Then do minimization of distances.
4) Generic mask-based SI: select some region, e.g., region around the tooth, and segment anatomy in it. Put a fine grid over this region. For each grid point, see which anatomical area is detected there. Then do SI by minimizing distances between all similar anatomical regions and maximizing distance between dissimilar ones.

A localizer-descriptor pipeline for segmentation of teeth+detection of pathologies for intraoral x-rays (bitewing, periapical, occlusal images) has also been developed. The pipeline is similar to the CBCT/OPT described above (localizer/descriptor pipeline). The pipeline may also be configured for segmentation of teeth+detection of pathologies for intraoral photography (optical-visible light-based). Developed also is a localizer-descriptor pipeline for segmentation of teeth+detection of pathologies for intraoral optical scans (3D surface obtained with highly precise laser depth sensor and configured for optical texture-visible light-display).

Developed is a module that creates 3D models from tooth and anatomy masks. It uses marching cubes algorithm followed by Laplacian smoothing to output a 3D mesh, which is later saved to STL. Certain anatomical objects can be grouped together to provide a surface containing selected objects (e.g., jaw+all teeth except one that is marked for extraction, for the purposes of a separate 3D-printing a surgical template for precise drilling of the implant hole).

Finally, a module that co-registers CBCT and IOS taken at the same time has been developed: Input is CBCT and IOS in separate coordinate systems. The output is IOS translated to coordinate system of CBCT. IOS have higher resolution, while CBCT displays the insides of the tooth/jaw. This is achieved by detecting the same dental landmarks (distinct points on teeth) on CBCT and IOS; at which point, minimize the distance between same points on CBCT and on IOS, which will give a coordinate transform to apply to IOS.

Advantageously, the present invention provides an end-to-end pipeline for detecting state or condition of the teeth in dental 3D CBCT scans. The condition of the teeth is detected by localizing each present tooth inside an image volume and predicting condition of the tooth from the volumetric image of a tooth and its surroundings. Further, the performance of the localization model allows to build a high-quality 2D panoramic reconstruction—with EoI focused-which provides a familiar and convenient way for a dentist to inspect a 3D CBCT image. The performance of the pipeline—with image processor, parsing engine, localization layer, EoI engine, and segmentation modules—is improved by adding i/v.i data augmentations during training; reformulating the localization task as instance segmentation instead of semantic segmentation; reformulating the localization task as object detection, and use of different class imbalance handling approaches for the classification model. Alternatively, the jaw region of interest is localized and extracted as a first step in the pipeline. The jaw region typically takes around 30% of the image/image volume and has adequate visual distinction. Extracting it with a shallow/small model would allow for larger downstream models. Further, the diagnostic coverage of the present invention extends from basic tooth conditions to other diagnostically relevant conditions and pathologies.

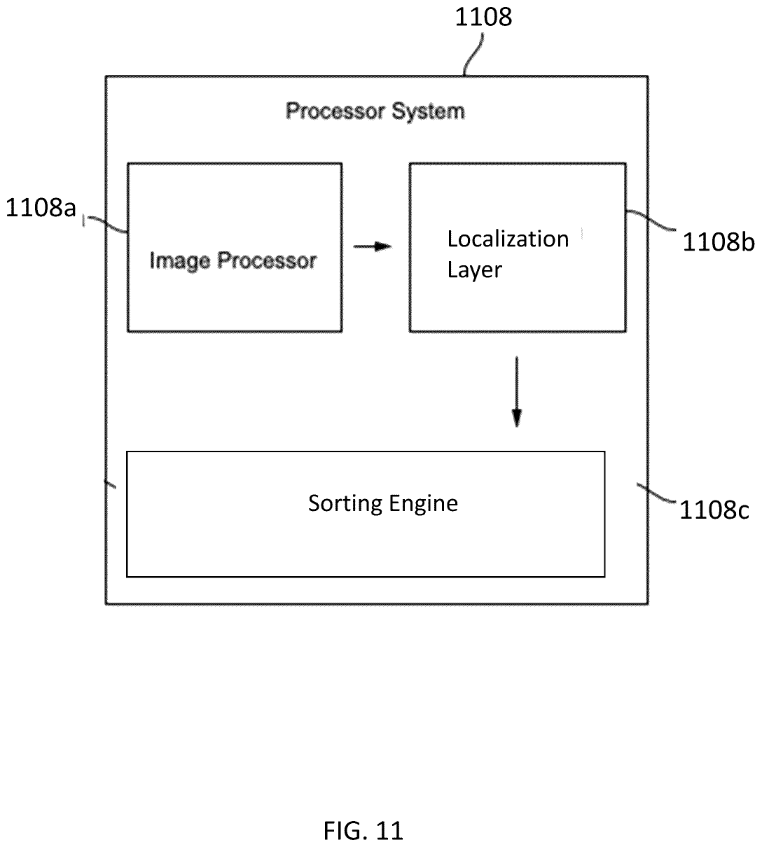

FIG. 11 illustrates in a block diagram, a localizer-descriptor pipeline for segmentation of teeth and (optionally) detection of pathologies for intraoral x-rays (bitewing, periapical, occlusal images) and/or panoramics. The localizer-descriptor pipeline, illustrated in system block form in FIG. 11, may also be referred to as an automated tooth localization and enumeration system in accordance with an aspect of the invention. The pipeline/system is similar to the CBCT/OPT described above (localizer/descriptor pipeline). The system may also be configured for segmentation of teeth+detection of pathologies for intraoral photography (optical-visible light-based). The segmentation of teeth+detection of pathologies for intraoral optical scans (3D surface obtained with highly precise laser depth sensor and configured for optical texture-visible light-display) may also be implemented by the pipeline/system as described herein and as illustrated in FIG. 11.

In an exemplary embodiment—as shown in FIG. 11—the system may comprise an input system, an output system, a memory system or unit, a processor system 1108, an input/output system and an interface. The processor system 1108 may comprise an image processor 1108a, (optionally) a parsing engine in communication with the image processor 1108a, a localization layer 1108b in communication with the parsing engine (optional) and/or image processor 1108a. The processor 1108 is configured to receive at least one 2-D image (radiographic or digital) via an input system. At least one received radiographic or digital 2-D image (2-D R/D) may, optionally, have its pixel array pre-processed to convert into an array of Hounsfield Unit (HU) radio intensity measurements. Then, the processor 1108 may optionally be configured to parse the received 2-D R/D into at least a single image frame field of view by the said image processor 1108a.

The anatomical structures residing in the at least single field of view or whole image is localized by assigning each pixel a distinct anatomical structure by the localization layer 1108b, or optionally, by the parsing engine. The processor 1108 or localization layer 1108b is configured to select all pixels belonging to the localized anatomical structure by finding a minimal bounding rectangle around the pixels and the surrounding region for cropping as a defined anatomical structure by the localization layer 1108b. In some embodiments. The pipeline/system may additionally segment pathologies on a 2-D R/D over an entire image and/or a cropped image of defined anatomical structure as defined by the localizer using a detection module/layer, such as a 2-D instance segmentation module (2-D R-CNN) (not shown).

FIG. 11 illustrates an exemplary automated tooth localization and enumeration system. The system may comprise: an image processor 1108a; a localization layer 1108b; a sorting engine 1108c; a processor 1108; a non-transitory storage element coupled to the processor; encoded instructions stored in the non-transitory storage element, wherein the encoded instructions when implemented by the processor 1108, configure the system to: receive a series of at least one of an intra-oral or panoramic images constituting a full mouth series (FMX) from a radio-image gathering or digital capturing source for processing by the image processor 1108a; parse the series of images into at least a single image frame field of view by said image processor 1108a; localize and enumerate (annotate) at least one tooth residing in the at least single image frame field of view by assigning each pixel a distinct tooth structure by selecting all pixels belonging to the localized tooth structure by finding a minimal bounding rectangle around the pixels and the surrounding region for cropping as a defined enumerated tooth structure image by the localization layer 1108b; and sort images using the defined enumerated tooth structure images to fill an FMX ((full mouth series) mounting table by the sorting engine 1108c.

In one embodiment, the localization layer 1108b includes 33 class semantic segmentation in 2-D. In one embodiment, the system is configured to classify each pixel as one of 32 teeth or background and resulting segmentation assigns each pixel to one of 33 classes. In another embodiment, the system is configured to classify each pixel as either tooth or other anatomical structure of interest. In case of localizing only teeth, the classification includes, but not limited to, 2 classes. Then individual instances of every class (teeth) could be split, e.g. by separately predicting a boundary between them. In some embodiments, the anatomical structure being localized, includes, but not limited to, teeth, upper and lower jaw bone, sinuses, lower jaw canal and joint.

In one embodiment, the system utilizes fully-convolutional network. In another embodiment, the system works on downscaled images and grayscale (1-channel) image (say, 1×100×100-dimensional tensor). In yet another embodiment, the system outputs 33-channel image (say, 33×100×100-dimensional tensor) that is interpreted as a probability distribution for non-tooth vs. each of 32 possible (for adult human) teeth, for every pixel.

In an alternative embodiment, the system provides 2-class segmentation, which includes labelling or classification, if the localization comprises tooth or not. The system additionally outputs assignment of each tooth pixel to a separate “tooth instance”.

In one embodiment, the system comprises FCN/CNN (such as U-Net) predicting multiple “energy levels”, which are later used to find boundaries. In another embodiment, a recurrent neural network could be used for step by step prediction of tooth, and keep track of the teeth that were outputted a step before. In yet another embodiment, Mask-R-CNN generalized to 2-D may be configured to take multiple crops from 2-D image in original resolution, perform instance segmentation, and then join crops to form mask for all original image. In another embodiment, the system could apply either segmentation or object detection in 2-D, to perform localization, enumeration or diagnostic functions. Furthermore, this would allow to process images in original resolution (albeit in 2D instead of 3D) and then infer 3D shape from a 2D segmentation.

In one embodiment, using any one of a pixel-level prediction technique, a tooth structure may be localized and enumerated within a cropped image from a full mouth series, wherein the condition is detected by at least one of detecting or segmenting a condition on at least one of the enumerated tooth structures within the cropped image. Localization may be improved by adding i/v.i data augmentations during training; reformulating the localization task as instance segmentation instead of semantic segmentation; reformulating the localization task as object detection, and use of different class imbalance handling approaches for the classification model. As a further method of improving localization, the jaw region of interest is localized and extracted as a first step in the pipeline. The jaw region typically takes around 30% of the image/image volume and has adequate visual distinction. Extracting it with a shallow/small model would allow for larger downstream models.

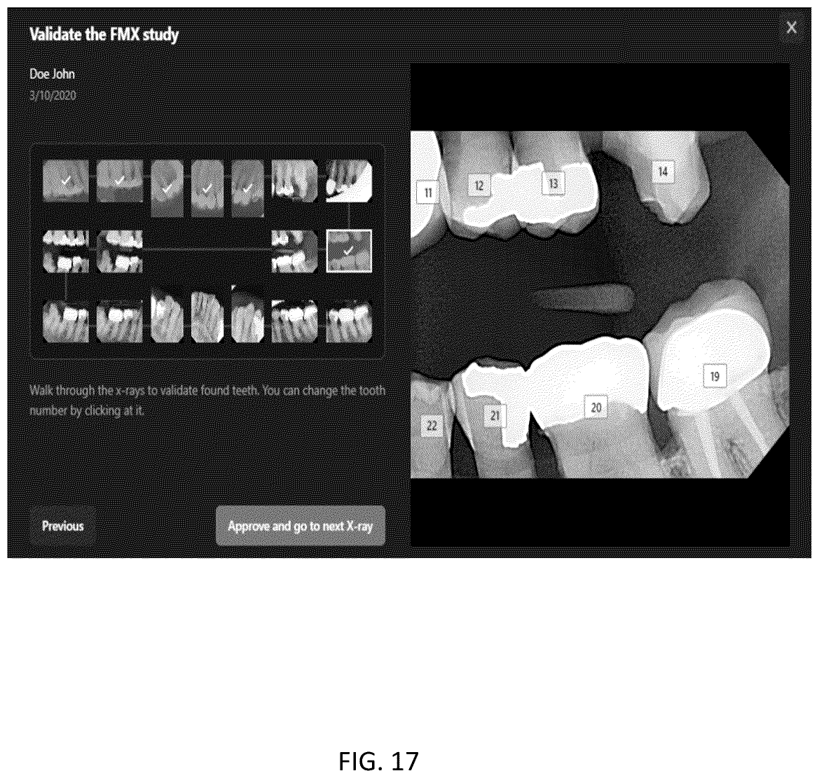

While not shown, the system may further comprise a parsing engine or module, wherein parsing is achieved by taking input images (2-D R/D) and transform the images to a gray-scale pixel intensity matrices, suitable for subsequent processing by a convolutional neural network (CNN) for downstream localization, enumeration, or condition detection. Localization may be achieved by performing object detection and/or subsequent semantic segmentation of any tooth using any kind of object detection CNN trained on a dataset of x-rays with teeth annotated using at least one of bounding boxes or pixel-wise masks. FIG. 17 illustrates an exemplary screen shot of a localized 2-D R/D image having been localized via the 2-D R-CNN-mediated object detection and/or semantic segmentation (localization layer). As FIG. 17 illustrates, the practitioner may click on any tooth/tooth number (localized/enumerated) for spotlight, validation, or further diagnostic prompting.

While not shown, the system may further comprise an enumeration layer, achieving enumeration by performing at least one of a direct classification approach, separate model classification branches, or a semantic segmentation sub-model. For example, in the first approach (direct classification), directly classify the number of tooth (1-52, 1-32 for permanent dentition, 33-52 for primary dentition). For the second approach (separate model classification), use separate model classification branches (heads, subunits) to identify: 1) anatomical tooth number (1-8); 2) whether the tooth is primary or permanent; 3) whether the tooth is live tooth or a tooth-like construct (implant, pontic, etc.); 4) whether the tooth is primary (child/milk tooth) or permanent; 5) classifying anatomical side (left/right); and 6) classifying jaw (maxilla/mandible). With respect to the third approach (semantic segmentation sub-model), follow the second approach, but replace step 4)-6) with semantic segmentation sub-model that predicts pixel assignment to dental chart quarters 1-8 (4 for permanent, 4 for primary) for all teeth.

Regardless of what approach is employed, one may perform enumeration or classification on the tooth crop (minimal bounding box of the tooth); or significantly extend the crop with a surrounding image as a context (to capture details that could help the model with identifying the tooth's dental quarter); or finally, perform classification on the whole image, while passing a binary mask of the tooth we mean to classify as an additional feature (input image channel).

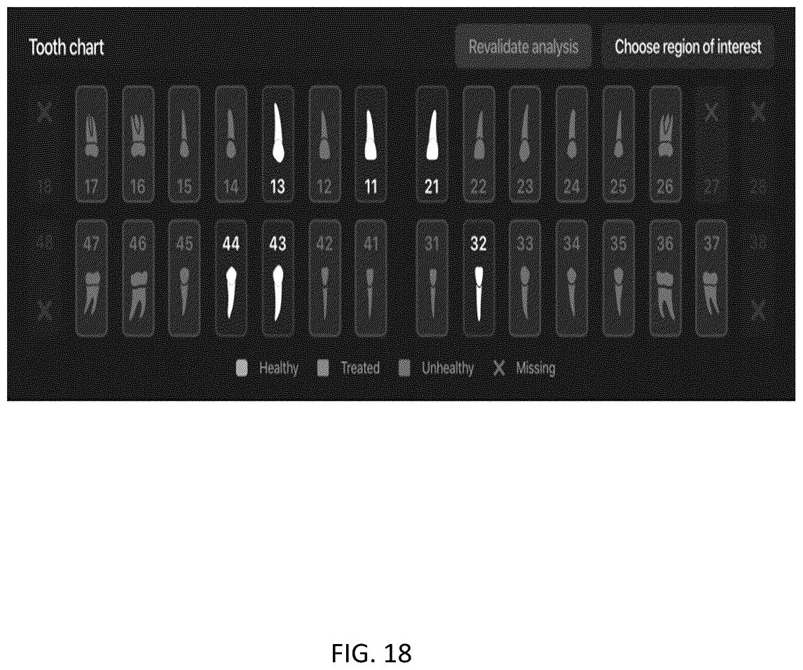

The system may further comprise an enumeration post-processing layer, achieving enumeration post-processing by receiving input of predicted tooth numbers; re-orienting the image using a correct orientation prediction by a separate classification neural network; partitioning predicted tooth numbers in correct order; and reassigning numbers of incorrect numbers to get tooth order consistent with a standard tooth chart. FIG. 18 illustrates in a screenshot, an example of a localized and annotated (enumerated) tooth/teeth in an embodiment of the present invention. More crucially, it also annotates for unhealthy teeth. For instance, based on the data result of the localization and enumeration, tooth 23 appears to be annotated as unhealthy which may prompt further diagnostic evaluation.

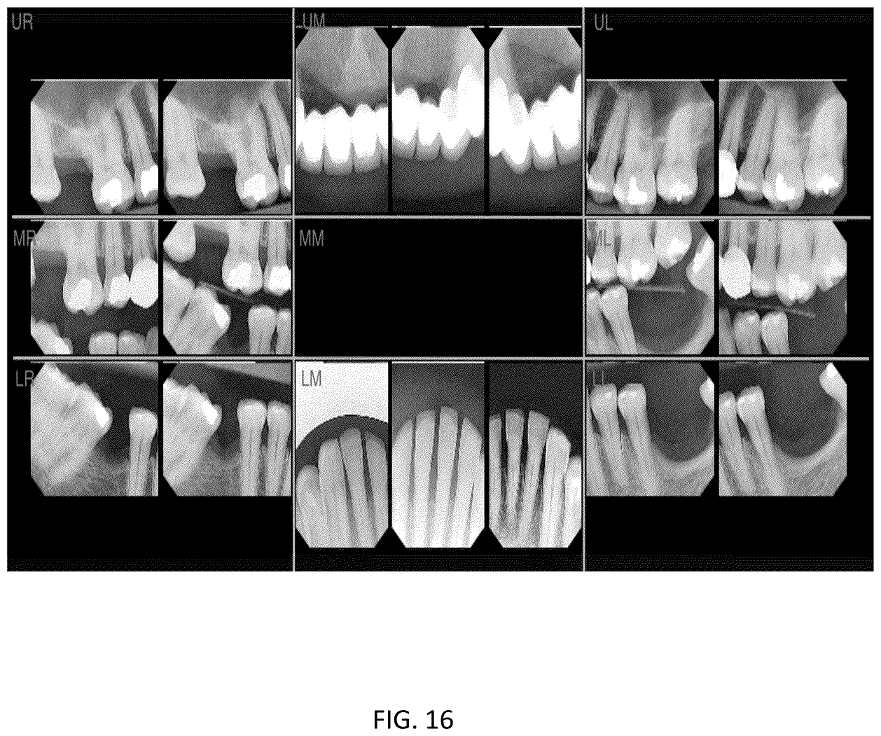

Furthermore, sorting may be achieved by the sorting engine 1108c as shown in FIG. 11. The sorting engine 1108c achieves sorting by first receiving average signed anatomical tooth number (from −8 to 8, ignoring the tooth quadrant) for each image; then partitioning images into nine location-based partitions: UR-UM-UL-MR-MM-ML-LR-LM-LL (as shown in FIG. 16 illustrating in a screenshot, an example of a sorted image) based on a set of anatomical tooth numbers identified on the image, where upper row receives images with only maxillary teeth, lower row (LR-LM-LL) receives images with only mandibular teeth, middle row receives images with a mix of maxillary and mandibular teeth; and patient-right (UR-MR-LR) side receives images that display only teeth from the right side of the patient face, patient-left (UL-ML-LL) side receives images that display only teeth from the left side of the patient face, and middle side (UM-MM-LM) receives images containing a mix of both left and right teeth; and then ordering and sorting images using that number in ascending order. While not shown, some embodiments additionally include a validation module, wherein validation is achieved by studying the preliminary matrix of numbered teeth, looking for teeth that are out of order, and if out of order, reassigning the tooth number and re-sorting the matrix. Validation may additionally be performed by the practitioner and requested to confirm prior to moving to next slide—as indicated in the lower left region of FIG. 17 and top right region of FIG. 18. In another embodiment, partitioning images between patient right, middle and left sides are achieved by establishing upper and lower bounds on average signed anatomical tooth number, such as values below −3 are assigned to patient-right side partitions; values between −3 and 3 are assigned to middle partitions; values above 3 are assigned to patient-left partitions, where three [3] is a constant that could be any number.

Furthermore, sorting is achieved by the sorting engine 1108c as shown in FIG. 11. The sorting engine 1108c achieves sorting by partitioning teeth into nine location-based partitions: UR-UM-UL-MR-MM-ML-LR-LM-LL; and then ordering within each partition by first receiving average signed anatomical tooth number for each side; and then sorting images using that number in ascending order. While not shown, some embodiments additionally include a validation module, wherein validation is achieved by studying the preliminary matrix of numbered teeth, looking for things that are out of order, and if out of order, reassigning the number and re-sorting the matrix. Validation may additionally be performed by the practitioner and requested to confirm prior to moving to next slide—as indicated in the lower left region of FIG. 17 and top right region of FIG. 18.

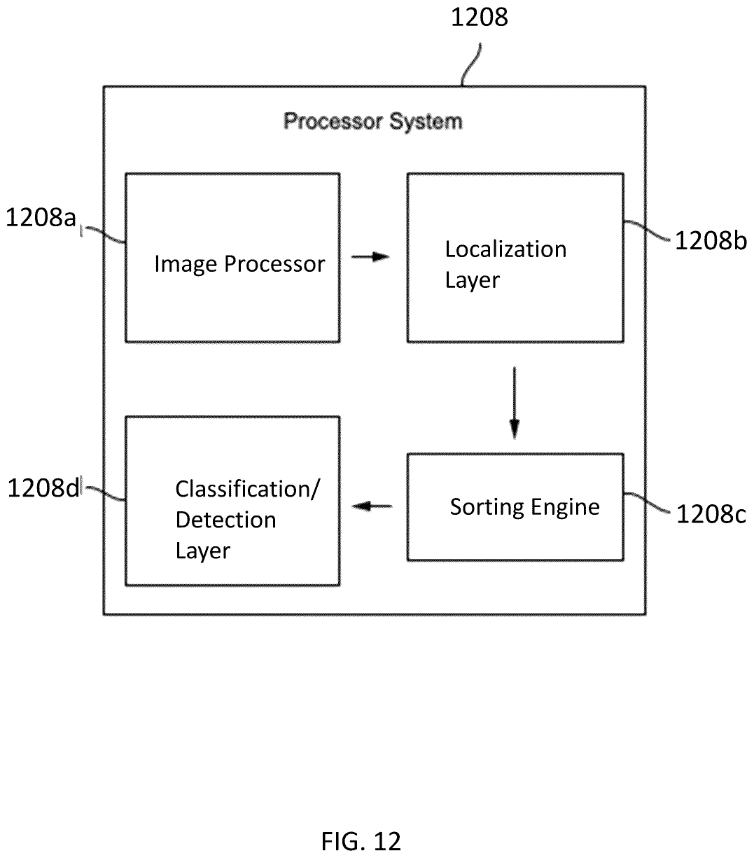

FIG. 12 illustrates in a block diagram, an automated system for localizing, enumerating, and diagnosing tooth conditions from dental images according to an embodiment. In one embodiment, the system may comprise: an image processor 1208a configured for processing at least one of an intra-oral or panoramic x-ray image; optionally, a parsing module/image processor to parse the series of images into at least a single image frame field of view by the image; a localization layer 1208b; optionally, a sorting engine using the defined enumerated tooth structure image to fill an FMX (full mouth series) mounting table; a classification/detection layer 1208d; a processor 1200; a non-transitory storage element coupled to the processor 1200; encoded instructions stored in the non-transitory storage element, wherein the encoded instructions when implemented by the processor 1200, configure the system to: receive a series of images constituting a full mouth series from a radio-image gathering or digital capturing source by the image processor 1208a; localize and enumerate at least one tooth residing in at least single image frame field of view by assigning each pixel a distinct tooth structure by selecting all pixels belonging to the localized tooth structure by finding a minimal bounding rectangle around said pixels and the surrounding region for cropping as a defined enumerated tooth structure image by the localization layer 1208b; optionally, sort the images to fill the FMX mounting table; and detect conditions for each defined enumerated tooth structure within a cropped image, wherein conditions are detected by the classification layer/module or detection layer/module 1208d, wherein the detection module 1208d at least one of detects or segments conditions and pathologies on at least one of the enumerated tooth structures within the cropped image.

In some embodiments, conditions may be detected by training a CNN to either detect (using object detection architectures) or segment (using multiple binary semantic segmentation architectures) conditions and pathologies on FMX, panoramics and various other crops with respect to partial or whole tooth/teeth. CNNs may be trained on many data sets containing different types of conditions in multi-task fashion. Each example is trained on only for conditions that are defined for it. Conditions that are not defined will be masked out (their per-condition loss is multiplied by 0 before back-propagating).

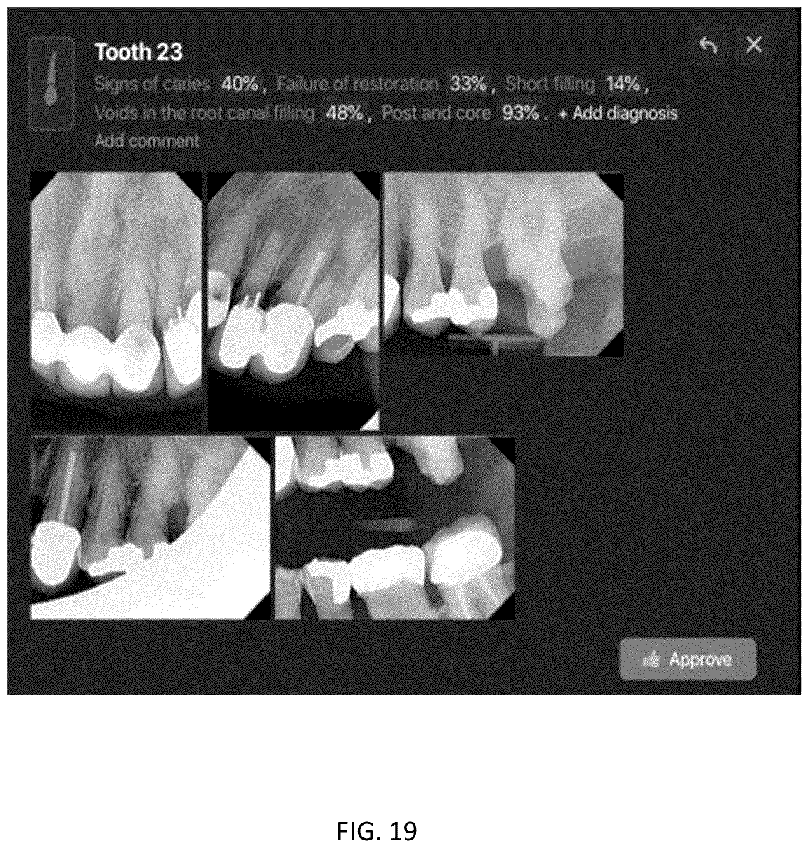

Some conditions may be related to whole tooth and are not localized to any specific area on the tooth. To detect such conditions, a standard full-image classification CNN on a tooth crop (with possible context extension) may be used for training. Examples of such conditions are: impacted tooth, dystonic tooth, (+degree of impaction), implant, pontic, etc. Further, the diagnostic coverage of the present invention extends from basic tooth conditions to other diagnostically relevant conditions and pathologies. FIG. 19 illustrates in a screenshot, an example of a localized, annotated, and diagnosed tooth/teeth in an embodiment of the present invention. It spotlights the unhealthy marked tooth 23 from FIG. 18, wherein the percentages or likelihood of certain conditions being present for tooth 23 are predicted. For example, tooth 23 has a 40% predictive likelihood of caries and a 48% predictive likelihood of having voids in a root canal filing.

In one embodiment, the system could be implemented utilizing descriptor learning in the multitask learning framework i.e., a single network learning to output predictions for multiple dental conditions. This could be achieved by balancing loss between tasks to make sure every class of every task have approximately same impact on the learning. The loss is balanced by maintaining a running average gradient that network receives from every class*task and normalizing it. Alternatively, descriptor learning could be achieved by teaching network on batches consisting data about a single condition (task) and sample examples into these batches in such a way that all classes will have same number of examples in batch (which is generally not possible in multitask setup). Further, standard data augmentation could be applied to tooth images to perform scale, crop, rotation, vertical flips. Then, combining all augmentations and final image resize to target dimensions in a single affine transform and apply all at once. In some embodiments, the system could use coarse segmentation mask from localizer as an input instead of tooth image. In some embodiments, the descriptor could be trained to output fine segmentation mask from some of the intermediate layers. In some embodiments, the descriptor could be trained to predict tooth number.

As an alternative to multitask learning approach, “one network per condition” could be employed, i.e. models for different conditions are completely separate models that share no parameters. Another alternative is to have a small shared base network and use separate subnetworks connected to this base network, responsible for specific conditions/diagnoses.

Similar to pathology localizers, a model has been developed that will segment pathologies on panoramic radiograph. It operates over the entire image or inside a tooth RoI (bounding box predicted by OPT localizer). Any type of 2D segmentation network can be used, e.g. UNet. After pathology segmentation is obtained, assign pathologies to tooth by selecting those that are inside predicted tooth mask or are immediately adjacent to it.

Furthermore, in one embodiment, the detection module 1208d may be coupled to a segmentation module (optionally, semantic segmentation modules or 2-D U-Nets), configured to specifically segment localized pathologies, like caries and periapical lesions. We use these segmentation to 1) estimate volume of the lesions to track it size dynamic over time; 2) create very specific visualizations (FIG. 18); 3) get a “second opinion” network for diagnostics. These networks could also be trained in multi-task fashion, where one network learns to segment multiple types of lesions. A classification or detection module 1208d could also be attached to the outputs of any network layer(s) to produce probabilities for lesion or whole-image. In case of whole-image classifier, it could be used to add cheaper weakly labeled data (single whole-image label “image contains lesions”/“image does not contain lesions”) to costly segmentation labels (assigning lesion/background to each voxel). These networks typically operate on RoI defined by tooth sub-volume, like the Descriptor. However, non-dental diseases such as tumors and cysts in the jaws may also be diagnosed. In those cases, different RoI, like RoI of the upper/lower jaw, may be used.

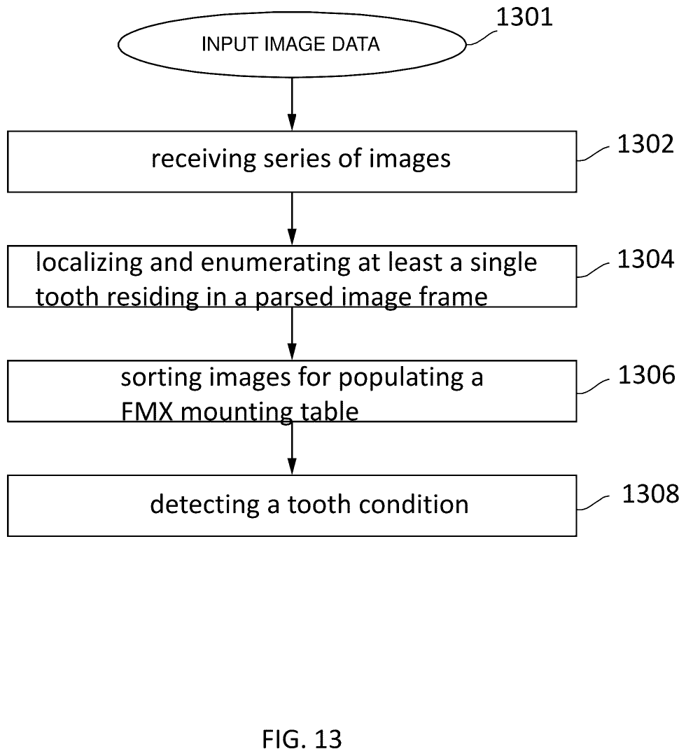

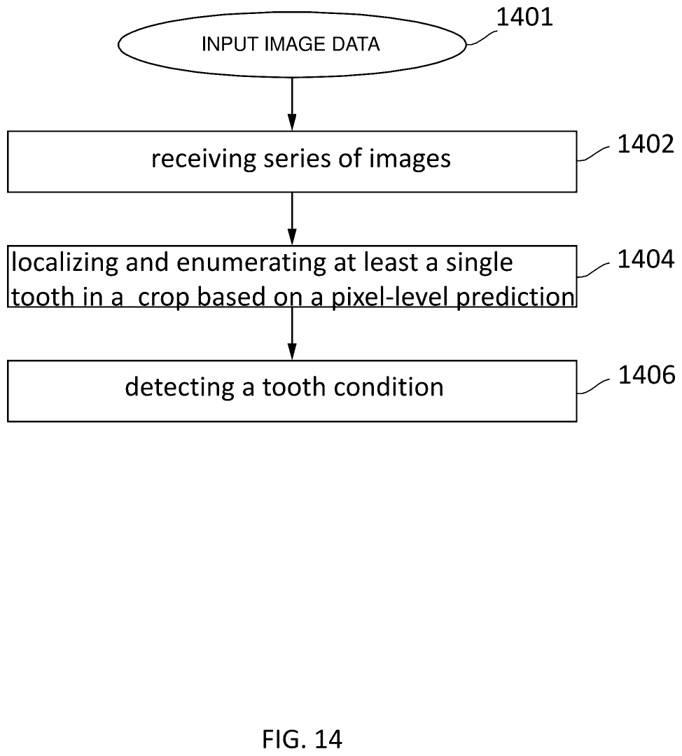

FIGS. 13 and 14 illustrate in method flow diagrams, an automated system for localizing, enumerating, and diagnosing tooth conditions from dental images according to an embodiment. FIG. 13 illustrates a method for tooth localization, enumeration, and diagnosing comprising the steps of: receiving a series of at least one of an intra-oral or panoramic images constituting a full mouth series from a radio-image gathering or digital capturing source for processing 1302; localizing and enumerating at least one tooth residing in at least single image frame field of view by assigning each pixel a distinct tooth structure by selecting all pixels belonging to the localized tooth structure by finding a minimal bounding rectangle around said pixels and the surrounding region for cropping as a defined enumerated tooth structure image 1304; sorting images using the defined enumerated tooth structure images to fill an FMX mounting table 1306; and detecting conditions for each defined enumerated tooth structure within a cropped image, wherein conditions are detected by at least one of detecting or segmenting conditions and pathologies on at least one of the enumerated tooth structures within the cropped image 1308.

FIG. 14 illustrates a method for automated localization, enumeration, and diagnosing of a tooth/condition. The method, as illustrated, comprises the steps of: receiving a series of at least one of an intra-oral or panoramic images constituting a full mouth series from a radio-image gathering or digital capturing source for processing 1402; localizing and enumerating at least one tooth structure residing in at least single cropped image based on a pixel-level prediction 1404; and detecting conditions for each defined enumerated tooth structure within the cropped image, wherein conditions are detected by at least one of detecting or segmenting conditions and pathologies on at least one of the enumerated tooth structures within the cropped image 1406.

In other embodiments, a method for automated localization and enumeration of a tooth is described. The method, while not illustrated, comprises the steps of: receiving a series of at least one of an intra-oral or panoramic images constituting a full mouth series from a radio-image gathering or digital capturing source for processing; and localizing and enumerating at least one tooth structure residing in at least single cropped image based on a pixel-level prediction.

The pixel-level prediction may be defined as any computer vision (C.V) task exploiting spatial redundancies in neighboring pixels resulting in image or object recognition/prediction based on an individual pixel within a broader pixel grouping (neighboring pixels). Examples of C.V. tasks include edge detection, object detection, convolutional networks, deep learning, semantic segmentation, etc.

Also, while not shown in FIG. 14, a method for automated localization, enumeration, and diagnosing of a tooth/condition may comprise the steps of: receiving a series of at least one of an intra-oral or panoramic images constituting a full mouth series from a radio-image gathering or digital capturing source for processing; and detecting conditions for each defined localized and enumerated tooth structure within a cropped image based on any one of a pixel-level prediction, wherein conditions are detected by at least one of detecting or segmenting conditions and pathologies on at least one of the enumerated tooth structures within the cropped image.

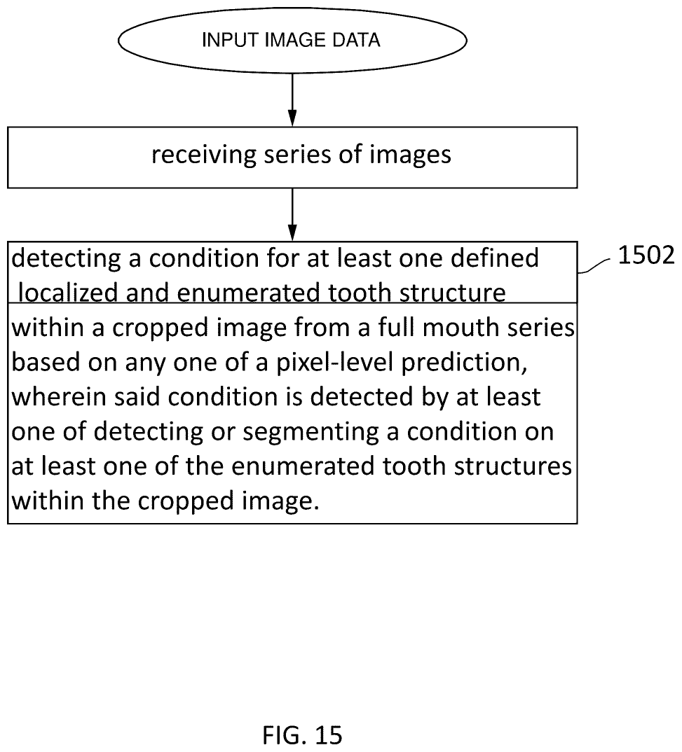

FIG. 15 illustrates a method for automated localization, enumeration, and diagnosing of a tooth/condition, said method comprising the step of: detecting a condition for at least one defined localized and enumerated tooth structure within a cropped image from a full mouth series based on any one of a pixel-level prediction, wherein said condition is detected by at least one of detecting or segmenting a condition on at least one of the enumerated tooth structures within the cropped image 1502.

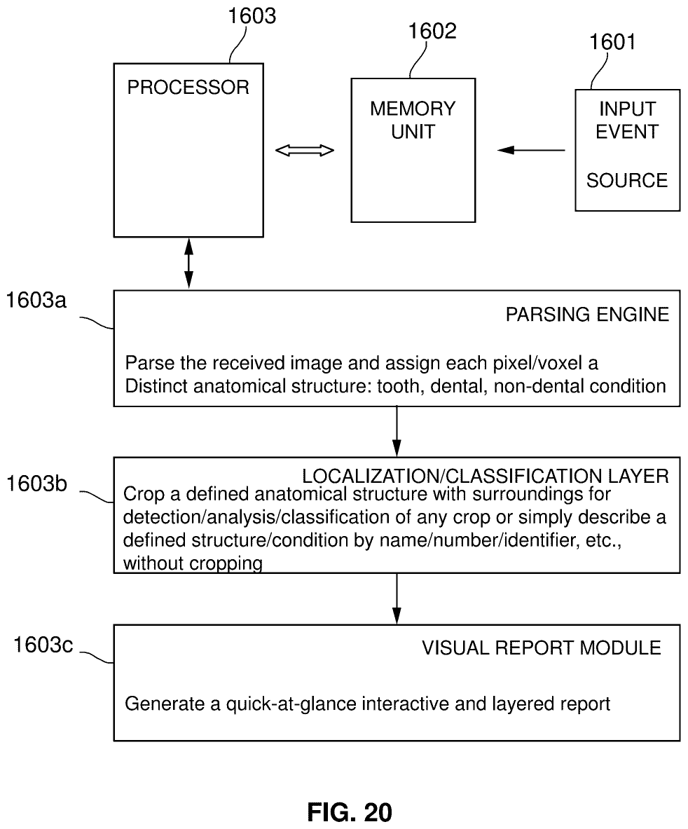

FIG. 20 illustrates a block diagram of the UI/visual layering system comprising an input event source 1601, a memory unit 1602 in communication with the input event source 1601, a processor 1603 in communication with the memory unit 1602, a pixel/voxel parsing engine 1603a in communication with a localizing/classifying layer 1603b. In an embodiment, the memory unit 1602 is a non-transitory storage element storing encoded information. The encoded instructions when implemented by the processor 1603, configure the UI/visual layering system to localize an anatomical structure and classify the structure/condition for quick-at-a-glance visual/informational/diagnostics for improved outcomes for a medical/dental practitioner—and patients likewise.

In one embodiment, an input data is provided via the input event source 1601. In one embodiment, the input data is a volumetric image data and the input event source 1601 is a radio-image gathering or CBCT image source. In one embodiment, the input data is a 2-Dimensional (2D) image data. In another embodiment, the input data is a 3-Dimensional (3D) image data. The processor 1603 is configured to receive the receive at least one image frame; localize at least one of a present tooth, dental, or non-dental condition inside the received image frame and identify it by at least one of a number, name, or short-hand; extract the at least one identified tooth, dental, or non-dental condition within the received image; classify the at least one tooth, dental, or non-dental condition based on the extracted; and represent results of the at least one classified area of interest in at least one of three layers, wherein the layers are an image-based layer, infographic-based layer, or an informational layer.

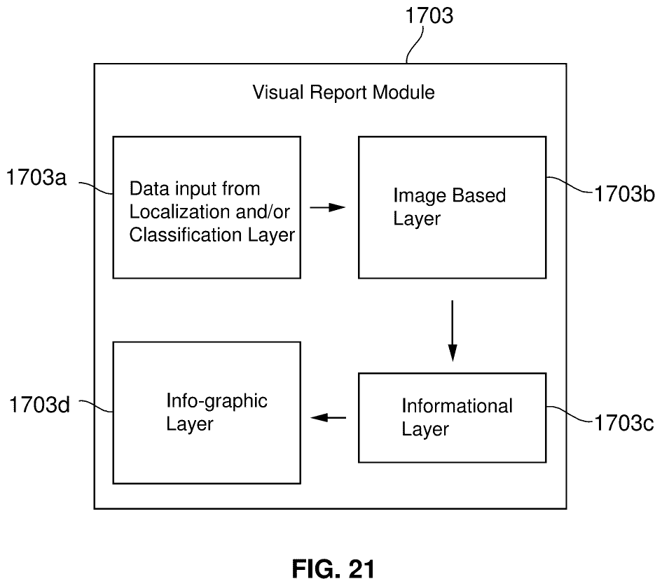

FIG. 21 illustrates an exemplary automated visual layering system (quick-glance interactive diagnostic reporting). The visual reporting module 1703 may comprise: a data input from a classification/localization layer 1703a; an image based layer 1703b; an informational layer 1703c; and an info-graphic layer 1703d; a processor; a non-transitory storage element coupled to the processor; encoded instructions stored in the non-transitory storage element, wherein the encoded instructions when implemented by the processor, configure the system to: receive a series of at least one of an intra-oral or panoramic images, or CBCT images; localize and enumerate (annotate) at least one tooth (or dental/non-dental condition) residing in the at least single image frame field of view by assigning each pixel/voxel a distinct tooth structure (condition) by selecting all pixels/voxels belonging to the localized tooth structure/condition by finding a minimal bounding rectangle around the pixels/voxels and the surrounding region for cropping as a defined enumerated image by the localization layer; and represent results of the enumerated tooth structure in at least one of three layers by the visual layering module, wherein the layers are an image-based layer 1703b, informational layer 1703c, and/or an infographic layer 1703d.

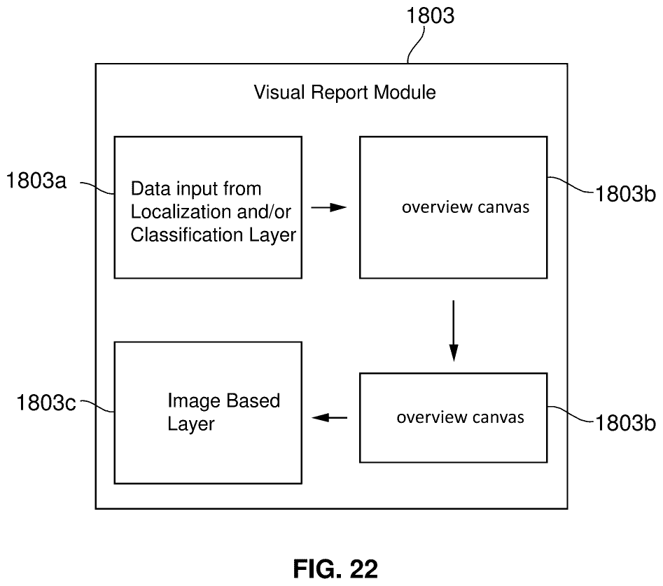

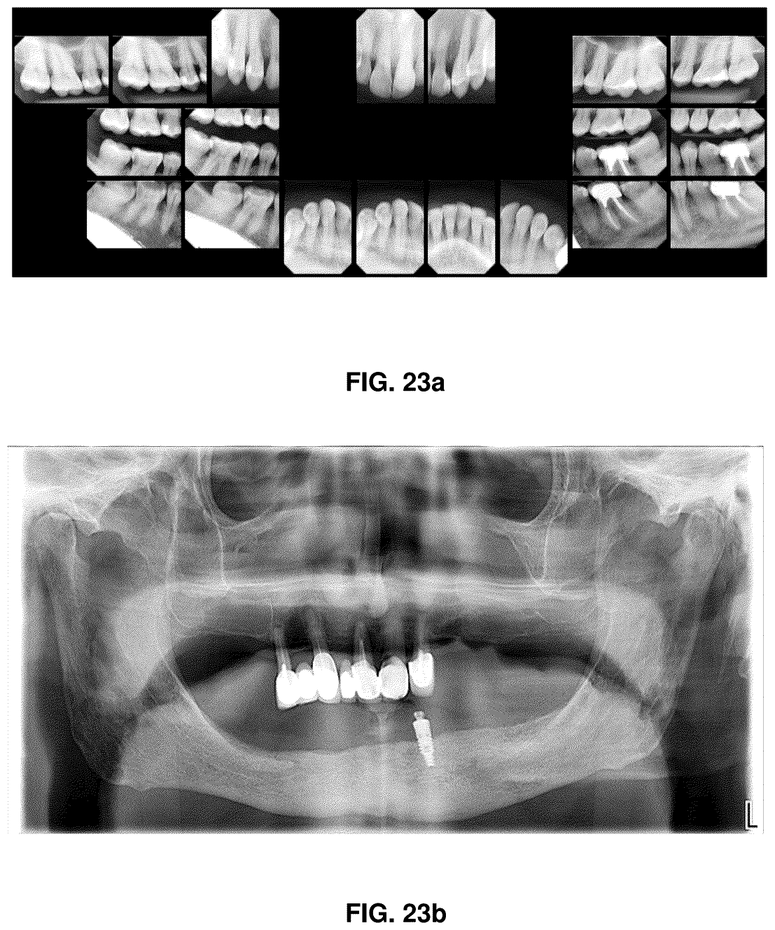

FIG. 22 illustrates a block diagram of the UI/visual layering system, particularly the image-based layer component of the visual reporting module 1803, comprising: a data input from classification/localization layer 1803a, a canvas overview 1803b/1803b and an image-based layer 1803c in communication with the input event source, a processor in communication with the memory unit, a pixel/voxel parsing engine in communication with a localizing/classifying layer. The image-based layer 1803c presents all of the pertinent data from the classification/localization layer 1803a into a visualization related to the image series as a whole. Referring to FIGS. 23A-23B, both are examples of an image series as a whole of a tooth sub-volume extraction presented via the image-based layer.

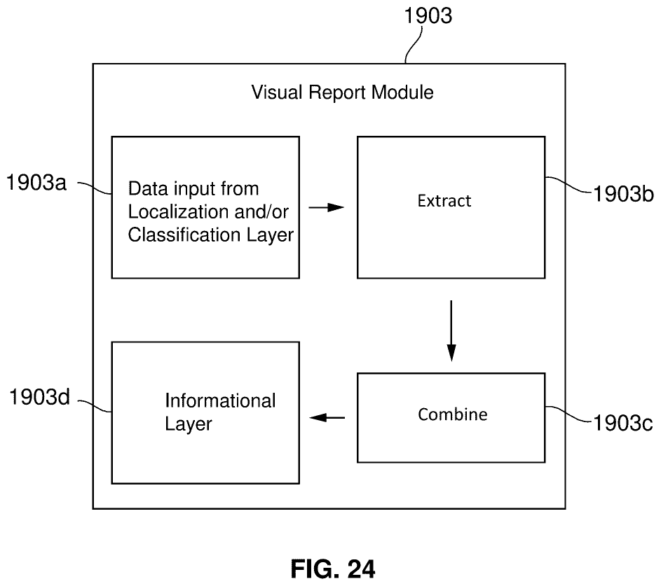

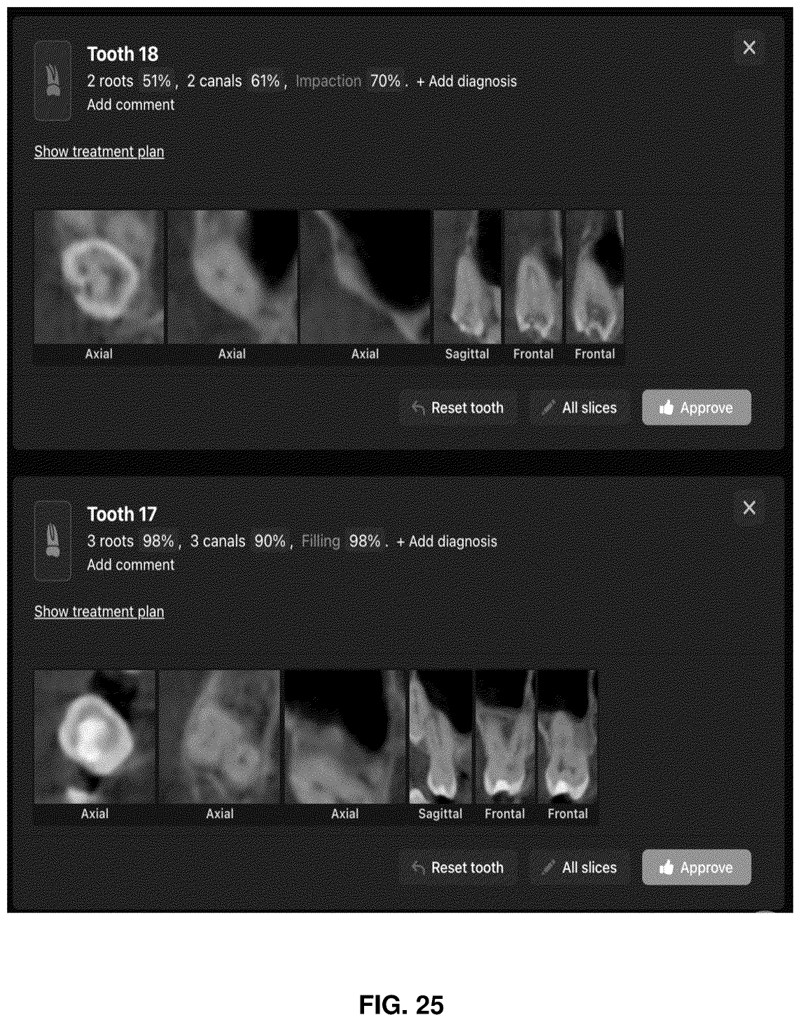

FIG. 24 illustrates a block diagram of the UI/visual layering system, particularly the informational layer component of the visual reporting module 1903, comprising: a data input from classification/localization layer 1903a, an extraction module 1903b, a combination module 1803c in communication with the input event source, a processor in communication with the memory unit, a pixel/voxel parsing engine in communication with a localizing/classifying layer. The informational layer 1803d presents all of the pertinent data from the classification/localization layer 1803a into an informative/pictographic related to the enumerated area of interest-extracting informationals and combining with diagnostic inferences. FIG. 25 illustrates the informational layer related to the analysis of teeth for CBCT image. More specifically, the screenshot displays informational elements for two teeth that were defined/enumerated. Each element presents: pictographic representation of tooth and its overall state; list of conditions that are detected (classified) for this tooth; multiple oblique slices taken from locations of interest that are considered interesting by AI.

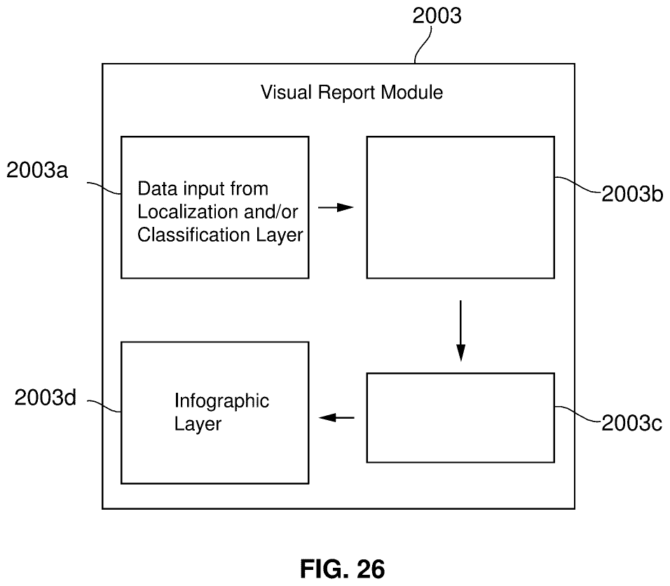

FIG. 26 illustrates a block diagram of the UI/visual layering system, particularly the infographic layer component of the visual reporting module 2003, comprising: a data input from classification/localization layer 2003a, a collection module 2003b; a presentation module 2003c in communication with the input event source, a processor in communication with the memory unit, a pixel/voxel parsing engine in communication with a localizing/classifying layer. The infographic layer 2003d presents all of the pertinent data from the classification/localization layer 2003a into a visually-rich interactive related to the enumerated area of interest. In one embodiment, the collection module 2003b collects the diagnostic data from classification/localization modules from all areas of interest classified/localized. The presentation module 2003c presents the at-a-glance overview of health status of the patient by taking a conventional pictographic/infographic representation of the areas of interest, such as tooth chart, and altering a pictogram/icon for each element by changing its color, style, shape, additional visual elements, etc.

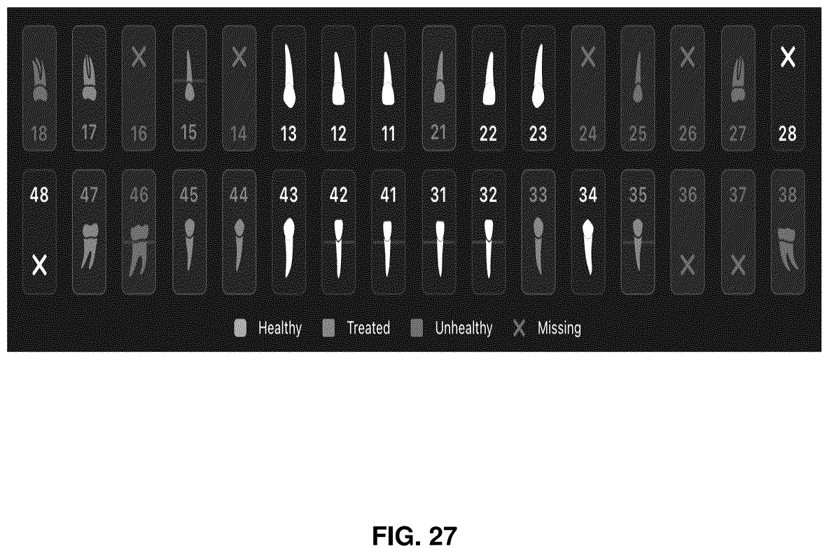

FIG. 27 illustrates an example of a localized and annotated (enumerated) tooth/teeth/condition in an embodiment of the infographic layer. Through the use of pictograms/icons/visuals/heuristics, a practitioner can very easily recognize tooth/teeth associated with any conditions. As shown, tooth 27 appears to be annotated as unhealthy, which may prompt further downstream diagnostic/treatment options. As shown in FIG. 27, the infographic layer may be seen as a visual summary for the state of all the teeth in patient's mouth. For instance, each tooth is colored according to its state, its shape is altered according to its number and condition, and any missing teeth are marked accordingly.

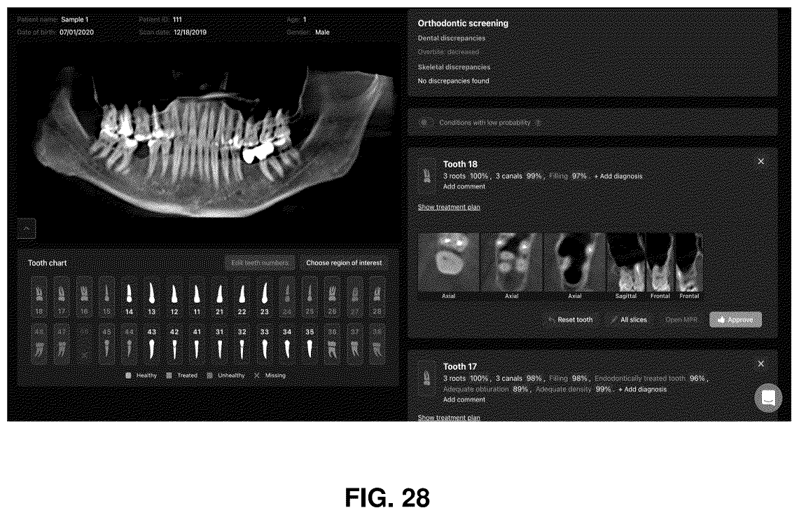

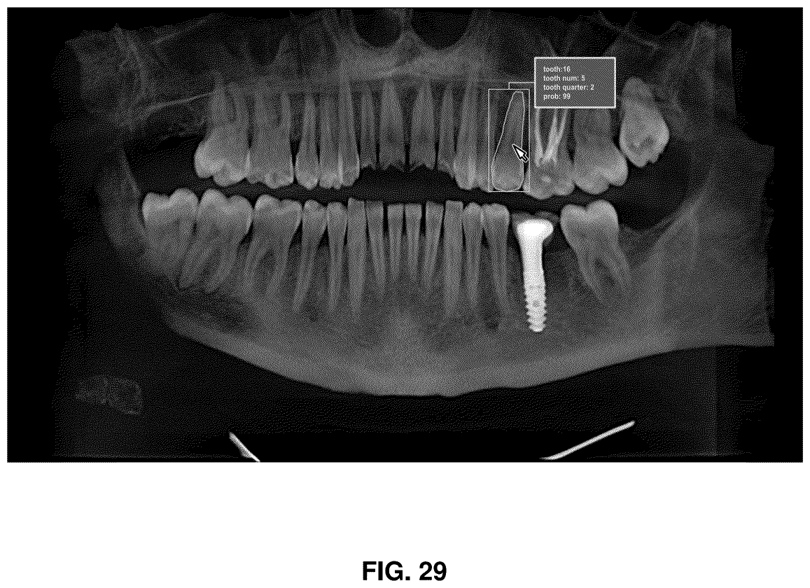

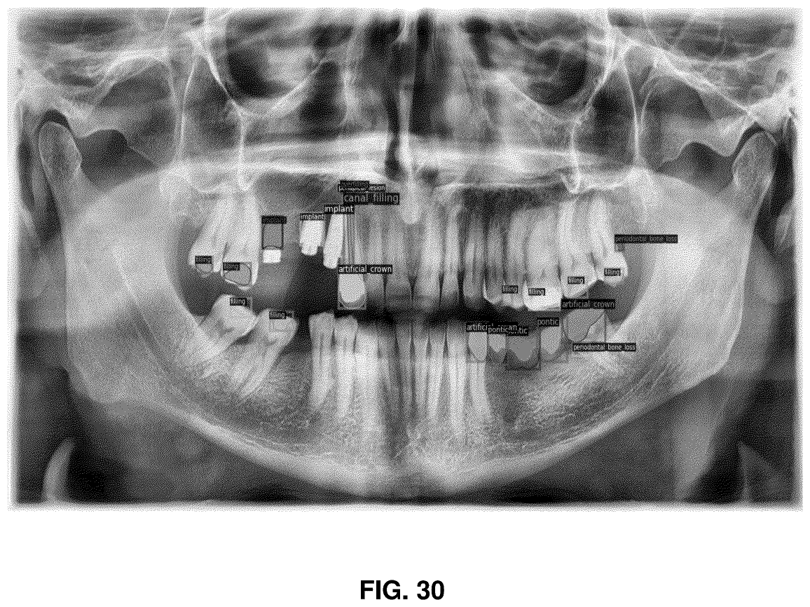

FIGS. 28-30 illustrate more subtle cursor control features enabling more seamless interaction within and between layers. FIG. 28, for instance, is an imposition of all three layers into a single display screen, enabling easier navigation between the layers by the practitioner. Upon noticing an interesting feature of the pano or CBCT image from the image-based layer, the practitioner may hover over a single area of interest for a quick informational or visual glimpse from any of the two layers. Upon hovering, and noticing an anomalous glimpse, a double click (or quick successive clicks) may take the practitioner to the corresponding layer. Under the same line of logic, a single click (or slow successive clicks) may take the practitioner to a quick informational or visual glimpse from another layer—with a double click routing the practitioner to the corresponding layer. Any combination of cursor control features may be possible for achieving the overall navigation within and between layers. FIG. 29 is a display of UI with per-object interactive areas: the area of interest (tooth) is outlined e.g. as a filled mask, contour, bounding box, or in other way; and the area of interest acts as an interaction anchor, e.g., a popup (quick informational/glimpse) may appear when the practitioner hovers over the area of interest (tooth 16, for instance). Finally, FIG. 30 is a combined visual representation of multiple findings of a single image, with each finding may be displayed as a filled mask, contour, bounding box, and/or text, etc.

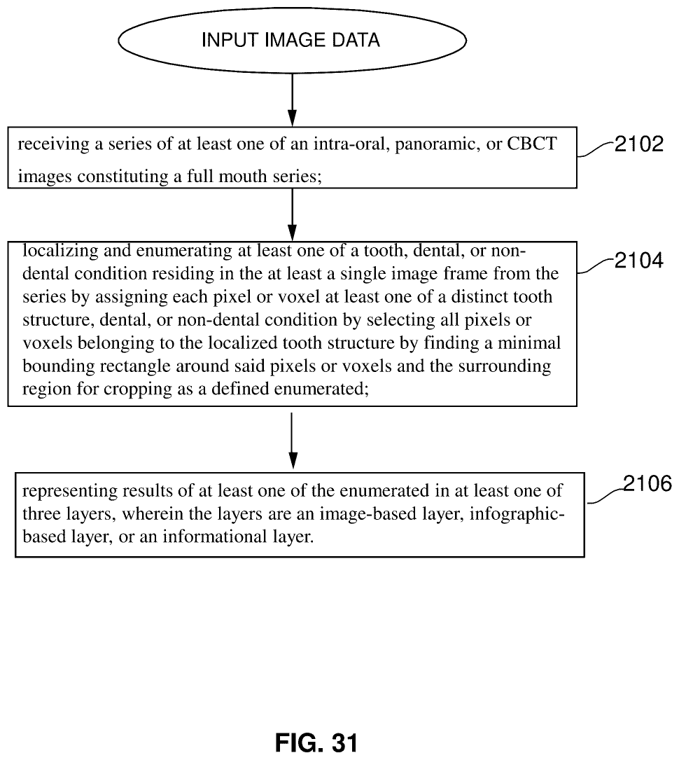

FIG. 31 illustrates a method flow for infographic display of an image analysis. In one embodiment, the method comprises the steps of: receiving a series of at least one of an intra-oral, panoramic, or CBCT images constituting a full mouth series 2102; localizing and enumerating at least one of a tooth, dental, or non-dental condition residing in the at least a single image frame from the series by assigning each pixel or voxel at least one of a distinct tooth structure, dental, or non-dental condition by selecting all pixels or voxels belonging to the localized tooth structure by finding a minimal bounding rectangle around said pixels or voxels and the surrounding region for cropping as a defined enumerated 2014; and representing results of at least one of the enumerated in at least one of three layers, wherein the layers are an image-based layer, infographic-based layer, or an informational layer 2106.

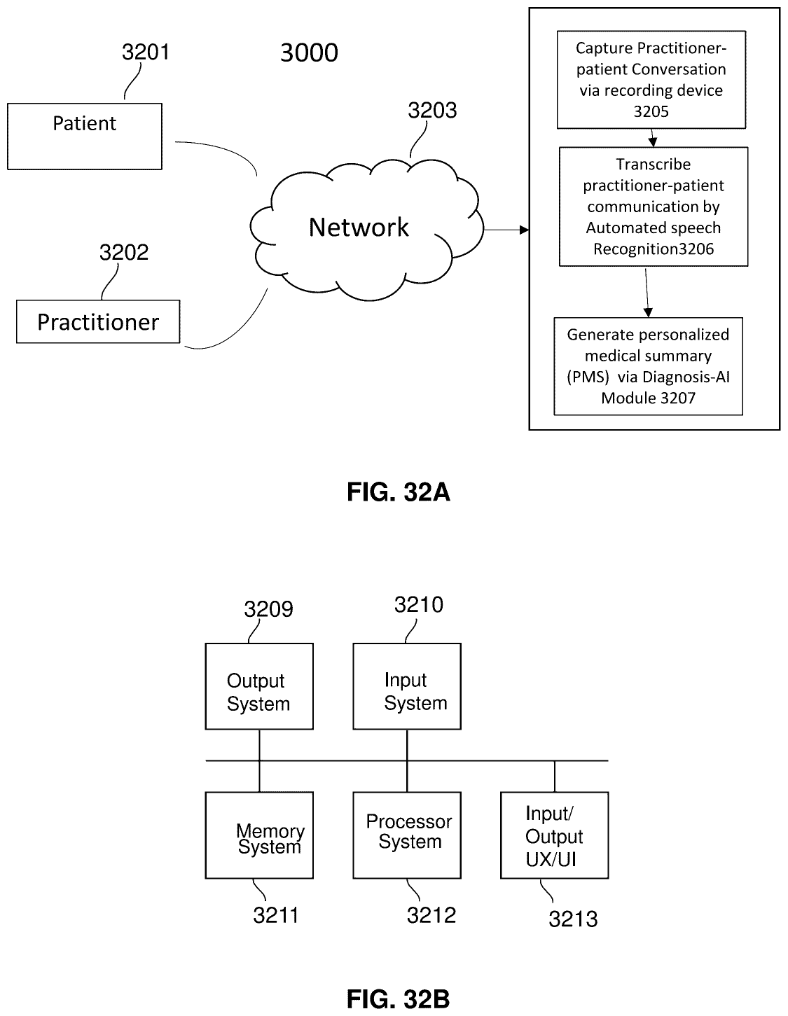

FIG. 32A and FIG. 32B illustrates a network diagram of a system to generate a personalized medical summary (PMS) from a practitioner-patient communication 3000. The networked environment includes a patient 3201, a practitioner 3202 and a diagnosis-AI (Artificial Intelligence) module (DAIM) 3207. The patient 3201 and practitioner 3202 and the DAIM 3207 are communicatively coupled through a network 3203. Typically, the DAIM 3207 generates a PMS based on the patient 3201 and a practitioner 3202 transcribed conversations which in turn enhances patient-practitioner communication.

In an exemplary embodiment, the network 3203 facilitates communication between a DAIM 3207, patient 3201, and the practitioner 3202 conversation. The practitioner-patient conversation 3205 is captured by a recording device. In an embodiment of the invention, the recording device is at least one of, but not limited to, voice recorders, smart phones, smart & digital devices, microphones, cameras, audio or video recorder, and digital transcription devices. Further yet, in an embodiment of the invention the practitioner-patient conversation is transcribed into a textual transcription via an automated speech recognition (ASR), wherein the ASR is at least one of, an off-the shelf, custom-built or a third-party service. Further yet, the ASR is performed by at least one of acoustic modeling-based ASR or neural network-based ASR. In a preferred embodiment of the invention, the PMS is rendered using at least one of a template-filling, do-by-example, and free-form summary format. More importantly, the DAIM may be integrated with a at least one of a conversational interface, chatbot or a voice-based AI-assistant wherein the DAIM integration is via by at least one of third-party API integration, file-based integration, screen scraping, and direct database integration. The DAIM suggests relevant information to the practitioner related to at least one of, potential diagnosis, treatment, planning, follow-up and communication with the patient. In yet another embodiment of the invention further comprises, a medical record storage module (MRSM) to record and save at least one of, patient data, previously generated PMS and past practitioner-patient conversations, wherein the patient data is at least one of current patient condition, patient dental/medical disease history, physical and mental health, past dental/medical treatments, X-rays/scans, medical complaints and list of medication.

In an embodiment of the invention, the patient 3201 communicates patient data and information related to symptoms, health concerns, and previous visits. The practitioner 3202 communication includes for visually-aided notes, diagnosis, recommendations, prescribed treatment, interactive maps and timelines. An intuitive input/output system, 3209, 3210 allows practitioners and patients to navigate the diagnosis modules. In another embodiment of the invention, the system may also incorporate a UX/UI 3213 to visualize a diagnosis report or a PMS, which provides a quick and efficient way for practitioners to input patient data and patients to respond to a query for an enhanced practitioner-patient communication. Examples of patient data inputs, but not limited to, symptom description, medical history, lifestyle factors, medication and supplement use, fears/concerns of diagnosis/treatments, expectations/goals regarding diagnosis/treatment. Further yet, patients may provide data via a real-time uploading of body worn devices typically embedded/equipped with one or more motion sensors, physiological sensors and environmental sensors. Examples of these sensors include, but are not limited to accelerometers, gyroscopes, inclinometers, geomagnetic sensors, global positioning systems, impact sensors, microphones, cameras, heart rate monitors, pulse oximeters, blood alcohol monitors, respiratory rate sensors, transdermal sensors, galvanic skin response (GSR) sensors and electromyography (EMG) sensors. In an embodiment of the present invention, the data captured by the one or more sensors is sent to the DAIM and, or the patient input through the network.

Typically, the body worn device is worn on one or more body parts of the patient, such as wrist, waist, neck, arm, leg, abdomen, chest, thigh, head, ear and fingers. Further, the body worn device may be a wristband, a watch, an armband, a necklace, a headband, an earring, a waist belt and a ring. The body worn device communicates with the mobile communication device (including, but not limited to, a smartphone, a tablet, a personal digital assistant (PDA), thin-client, and a mobile phone) over a short-range wireless communication medium. Examples of the short-range wireless communication medium include Bluetooth, ZigBee, Infrared, Near Field Communication (NFC) and Radio-frequency identification (RFID). Additionally, examples of practitioner-fed patient data include, but not limited to, diagnosis, treatment, planning and follow-up of dental/medical procedures.

In a continuing reference, the network may be any suitable wired network, wireless network, a combination of these or any other conventional network, without limiting the scope of the present invention. Few examples may include a LAN or wireless LAN connection, an Internet connection, a point-to-point connection, or other network connection and combinations thereof. The network may be any other type of network that is capable of transmitting or receiving data to/from host computers, personal devices, mobile phone applications, video/image capturing devices, video/image servers, or any other electronic devices. Further, the network is capable of transmitting/sending data between the mentioned devices. Additionally, the network may be a local, regional, or global communication network, for example, an enterprise telecommunication network, the Internet, a global mobile communication network, or any combination of similar networks. The network may be a combination of an enterprise network (or the Internet) and a cellular network, in which case, suitable systems and methods are employed to seamlessly communicate between the two networks. In such cases, a mobile switching gateway may be utilized to communicate with a computer network gateway to pass data between the two networks. The network may include any software, hardware, or computer applications that can provide a medium to exchange signals or data in any of the formats known in the art, related art, or developed later.

Further yet in an embodiment of the invention includes a memory system 3211 for storing dental/medical data to include, but not limited to, 1. Patient demographics such as name, age, gender, address, contact details, and insurance information, 2. Medical history to include past and current medical conditions, previous surgeries, allergies, medications, and family medical history, 3. Dental records to include information on dental examinations, diagnoses, treatments, and outcomes, as well as dental x-rays, CBCT scans, impressions, and photographs, 4. Vital signs to include measurements such as blood pressure, heart rate, respiratory rate, temperature, and oxygen saturation, 5. Laboratory results to includes blood tests, urine tests, and other diagnostic tests, which provide information on various health parameters, such as blood glucose levels, cholesterol levels, and liver function, 6. Imaging studies to include radiology reports, such as X-rays, CT scans, MRI scans, and ultrasound reports, which help in diagnosing and monitoring various conditions, 7. Prescription and medication information to include information on prescribed medications, dosage, frequency, and duration of use, 8. Surgical and procedural data to include information on surgeries, procedures, and interventions performed, including details such as date, type of procedure, surgical notes, and anesthesia used and, 9. Practitioner progress notes to include documentation of healthcare providers' observations, assessments, diagnoses, and treatments during patient visits, including dental and medical progress notes. The processor system 3212 is configured to process the stored data and generate a PMS via the DAIM.

The practitioner-patient communication is recorded 3205 via a recording device which may be at least one of, stationary, mounted or portable in the practitioner office/hospital/medical environment. The recording device is at least one of voice recorders, smart phones, smart & digital devices, microphones, cameras, audio or video recorder, and digital transcription devices.

In an embodiment of the invention, the system further comprises of an automated speech recognition (ASR) to convert the recorded practitioner-patient communication into textual transcription 3206. The ASR may be at least one of an off-the-shelf, custom-built or a third-party service and may be integrated into the PMS system directly or linked to a cloud-based platform. Further yet, in an embodiment of the invention, the ASR may be performed either using acoustic modeling-based ASR or neural network-based ASR. The acoustic modeling-based ASR or a neural network-based ASR may be off the shelf or custom designed. Acoustic modeling-based ASR systems are more accurate, but slower to develop and more computationally expensive whereas, neural network-based ASR systems are faster to develop and less computationally expensive, but less accurate. Further yet, in an embodiment of the system, a hybrid approach may be used to combine the advantages of acoustic modeling-based ASR and neural network-based ASR. For example—consider the practitioner and patient have a conversation/communication in a medical setting. The conversation is recorded using a voice recorder or other audio recording device. The audio recording is then uploaded to a cloud-based platform. The cloud-based platform uses a hybrid ASR system to transcribe the audio recording. The ASR system first uses an acoustic modeling-based system to generate a hypothesis of the words that were spoken between the practitioner and patient. This hypothesis is then passed to a neural network-based system, which refines the hypothesis and produces the final transcription of the practitioner-patient communication. Additionally, in an embodiment of the invention, the transcription of the practitioner-patient communication may be performed using an ASR specifically trained to transcribe medical conversations.

Further yet, in an embodiment of the invention the PMS is generated based on the transcribed conversation achieved by extracting clinically relevant information using at least one of a general-purpose large language model (LLM), a fine-tuned LLM trained for medical conversation or a custom-built LLM and is rendered using at least one of a template-filling, do-by-example, and free-form summary format. In an embodiment of the invention any commercially available LLM, such as but not limited to, ChatGPT, Bard, etc. may be integrated into the PMS system.

Further yet, a general purpose LLM generates a free-form summary of the transcript, which may include the patient's symptoms, diagnosis, and treatment plan. The summary would be written in a natural language and is easy for the practitioner and patient to understand. Below is an example of a prompt that may be used to generate a free-form summary of a transcript:

Patient: I've been having a lot of pain in my stomach.
Doctor: What kind of pain is it?
Patient: It's a sharp pain that comes and goes.
Doctor: Have you had any other symptoms?
Patient: Yes, I've also been feeling nauseous and vomiting.
Doctor: I think you might have appendicitis.
I'm going to order an ultrasound to confirm
the diagnosis.

In another embodiment of the invention, an LLM may extract facts in a JSON (a machine-readable format that is commonly used to store data) format which may be directly imported into a medical records system.

{
“patient_name”: “John Doe”,
“age”: 35,
“sex”: “Male”,
“symptoms”: [“abdominal pain”, “nausea”, “vomiting”],
“diagnosis”: “Appendicitis”,
“treatment_plan”: [“surgery to remove the appendix”]
}

In a preferred embodiment of the invention a fine-tuned LLM may be used to generate a PMS of a patient via the DAIM (Diagnosis AI-Module). The DAIM is a specific dataset of medical and dental transcripts. A fine-tuned LLM is trained using DAIM to generate a more specific summary, such as a personalized medical summary. This allows the LLM to learn the specific language and terminology used in medical and dental records. Below is an example of a personalized medical summary that would be generated by the fine-tuned LLM using

```
DAIM-
  Patient: John Doe
  Age: 35
  Sex: Male
  Symptoms: Abdominal pain, nausea, vomiting
  Diagnosis: Appendicitis
  Treatment plan: Surgery to remove the appendix
```

In yet another embodiment of the invention, the DAIM is integrated with a at least one of a conversational interface, chatbot or a voice-based AI-assistant. The DAIM integration is via by at least one of third-party API integration, file-based integration, screen scraping, and direct database integration. Further yet, in an embodiment, third-party API integration allows for real-time data exchange which enables the conversation interfaces to send user inputs (patient or practitioner) or queries to the DAIM, which then processes the information and provides accurate diagnostic insights or recommendations back to the platform. In a file-based integration, the data (medical and dental data provided by the patient and practitioner) is exchanged between the DAIM and the conversational platform through files. The conversational interface sends relevant data in a structured file format to the DAIM, which then analyzes the information and generates diagnostic outputs. These outputs are subsequently sent back to the platform through files, allowing for seamless integration and communication between the two systems. In yet another embodiment of the invention, screen scraping based integration involves for extracting relevant data from the conversational interface's user (patient/practitioner) interface or screen. The DAIM further analyzes the extracted data to generate diagnostic information, which is in turn displayed or communicated back to the user (patient/practitioner) through the conversational platform-thus enabling integration even when direct API or file-based integration is either not available or feasible.

In yet another embodiment of the invention, a direct database integration between the DAIM (Diagnosis AI-Module) and a conversational platform involves establishing a direct connection between the databases of both systems-enabling a deeper level of integration, facilitating the exchange of information in real-time. The DAIM's database contains a wealth of medical/dental knowledge, diagnostic algorithms, and patient data and is directly connected to the conversational platform's database, which allows the conversational interface to access the DAIM's database and retrieve relevant diagnostic information as needed. The conversational database may be any large language models, such as ChatGPT, Neeva, Bard etc. Through this method, the conversational interface can directly access the DAIM's database to retrieve diagnostic information and present it to the user in real-time-thus, enhancing the accuracy, efficiency, and personalization of diagnoses/medical summary within the conversational interface, facilitating seamless and context-aware healthcare support.

In yet another embodiment of the invention, the DAIM suggests relevant information to the practitioner related to at least one of, potential diagnosis, treatment, planning, follow-up and communication with the patient. Further yet in the embodiment of the PMS system, the DAIM analyzes the transcribed conversation and clinical data to identify potential diagnoses by taking into account the symptoms discussed, medical history, and any additional information provided by the patient. Based on this analysis, the DAIM may suggest possible diagnoses for consideration by the practitioner which, may help the practitioner in formulating a differential diagnosis and considering appropriate treatment options. Further yet, regarding treatment suggestions—the DAIM may provide suggestions regarding suitable treatment options based on the identified diagnosis by taking into account evidence-based guidelines, best practices, and the patient's specific circumstances. The system may offer recommendations for medication, therapy, lifestyle modifications, or other interventions that align with the identified diagnosis. The suggestions may assist the practitioner in developing a personalized treatment plan for the patient. In another embodiment of the invention, Planning—The DAIM may aid in the planning phase of patient care by suggesting additional tests, imaging studies, or laboratory investigations that may be relevant for confirming or ruling out potential diagnoses. The PMS system may further consider the patient's symptoms, medical history, and the practitioner's assessment to recommend appropriate investigations—thus, ensuring a comprehensive and targeted approach to patient management.

In another embodiment of the invention, Follow-up—The DAIM may provide recommendations for follow-up actions based on the patient's condition and the chosen treatment plan. It may suggest a timeline for follow-up appointments, monitoring parameters to track progress, or specific instructions for patient self-care, thus helping the practitioner ensure an appropriate follow-up care and evaluate the effectiveness of the chosen treatment interventions. Additionally, in another embodiment of the invention, Communication with the Patient—The DAIM may also offer suggestions for effective communication with the patient by providing guidance on explaining the diagnosis, treatment options, and prognosis in a clear and understandable manner. Additionally, it may also suggest appropriate language or strategies for addressing any concerns or questions the patient may have thus, facilitating effective communication and patient engagement.

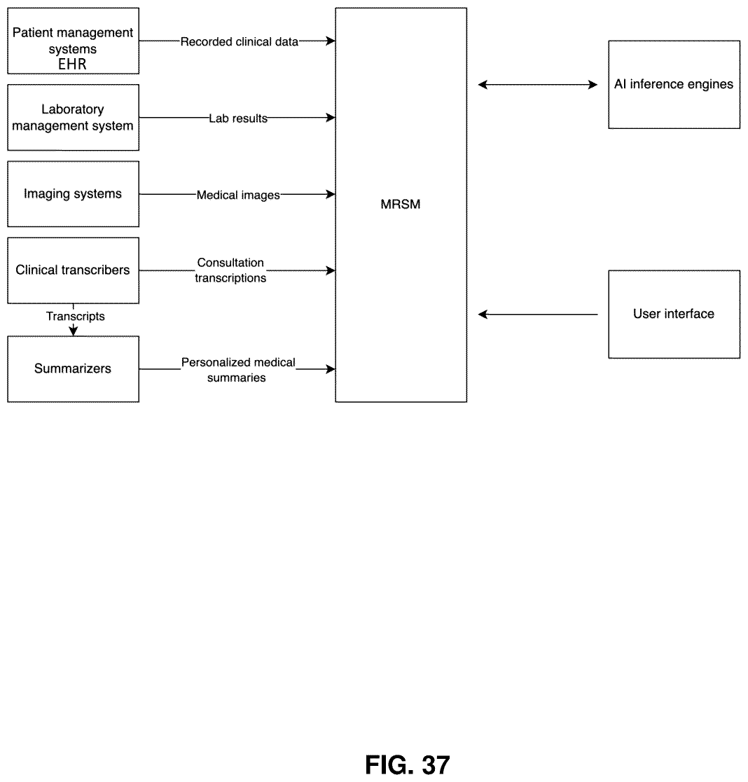

Now in reference, to FIG. 37, in yet another embodiment of the invention, the PMS system further comprises of a medical record storage module (MRSM) to record and save at least one of, patient data, previously generated PMS and past practitioner-patient conversations. Further yet, the patient data is at least one of current patient condition, patient dental/medical disease history, physical and mental health, past dental/medical treatments, X-rays/scans, medical complaints and list of medication.

In an embodiment of the invention FIG. 37, the MRSM stores essential patient data, which may include demographic information, medical history, allergies, current medications, and any other relevant details thus, providing a comprehensive overview of the patient's health profile and enables the healthcare providers (practitioners, nurses, nurse-aids etc.) to access and review pertinent information quickly. Further yet, in an embodiment of the invention, the MRSM may store information related to the patient's current health status to include details about their ongoing medical condition, such as symptoms, vital signs, laboratory test results, and imaging findings and provides a snapshot of the patient's present health and assists healthcare providers in assessing the current state of the patient's well-being. Additionally, the MRSM may record the patient's dental or medical disease history and capture information about any previous diagnoses, treatments, surgeries, or procedures the patient has undergone thus, helping healthcare providers understand the patient's medical background, identify any chronic conditions, and evaluate the impact of past treatments on the current health status. Further yet, the MRSM may include information related to the patient's physical and mental health which may encompass aspects such as pre-existing medical conditions, allergies, immunization records, mental health history, and psychiatric diagnoses. By storing this data, healthcare providers gain a comprehensive view of the patient's overall health and can tailor their approach accordingly. Additionally, the MRSM may retain records of past dental or medical treatments the patient has received, which may include details about specific procedures, medications administered, surgical interventions, and therapeutic interventions. This data assists in tracking the patient's treatment history, assessing the effectiveness of prior interventions, and informing future treatment decisions. Moreover, the MRSM may provide storage for medical imaging data, including X-rays, CT scans, MRIs, and other diagnostic images. Storing these images within the MRSM allows healthcare providers to access and review them when needed thus—aiding in diagnosing conditions, monitoring progress, and comparing current images with previous ones for comparison. Further yet, the MRSM may record patient-reported complaints, symptoms, and concerns, which may help the healthcare providers understand the patient's perspective and guides them in addressing specific issues during consultations. It may also be valuable for tracking the progression of symptoms and evaluating treatment outcomes. Additionally, the MRSM may maintain a list of medications the patient is currently taking, including prescribed medications, over-the-counter drugs, and supplements which, helps healthcare providers avoid potential drug interactions, monitor medication adherence, and consider any medication-related factors in the patient's overall treatment plan.

Further yet, the MRSM may also retain previously generated PMS summaries for each patient allowing healthcare providers to access historical PMS reports and review past diagnoses, treatments, and patient progress. Thus, access to the patient's PMS history helps healthcare providers track the patient's medical journey, make informed decisions, and ensure continuity of care. Additionally, the MRSM may store records of past conversations between practitioners and patients. These conversation records may include audio or textual transcripts of consultations, notes, and any other relevant documentation. Retaining these records enables healthcare providers to refer back to previous discussions, recall important details, and maintain a comprehensive record of the patient's healthcare interactions.

Further yet, the medical record storage module (MRSM) as shown in FIG. 37, is securely encrypted to ensure the privacy and confidentiality of patient data. The MRSM further comprising a search functionality within the medical record storage module (MRSM) to retrieve and display relevant patient information during the practitioner-patient conversation and is integrated with electronic health record (EHR) systems to synchronize and update patient data. Additionally, the MRSM may securely store and organize patient data, including medical history, current conditions, treatments, diagnostic results, and medication lists and have access to comprehensive and up-to-date patient information that allows healthcare providers to make informed decisions and provide appropriate care. Additionally, the MRSM may be linked to laboratory management system as well as imagining systems for storing, view of patient laboratory results and medical images including, but not limited to, X-rays, CT scans etc. Further yet, the MRSM may incorporate security measures to protect patient data, ensuring compliance with privacy regulations. By securely storing and encrypting medical records, the MRSM minimizes the risk of unauthorized access, data breaches, or loss of sensitive patient information which helps to maintain patient confidentiality. Further yet, the MRSM can integrate with electronic health records (EHR) systems, lab management systems, imaging systems, clinical transcribers and summarizers, facilitating seamless data exchange and interoperability between different healthcare providers. This integration enables the sharing of patient information across healthcare settings, promoting care coordination, and reducing duplication of tests or procedures. Additionally, in an embodiment of the invention, the clinical transcribers would transcribe transcripts into medical/dental summaries. The MSRM may be in a two way interaction with various AI inference engines as well as user interfaces.

Consider a scenario for the PMS system where Dr. Smith is a general practitioner uses the personalized medical summary (PMS) system during a patient consultation. The system comprises a processor, a Diagnosis-AI Module (DAIM), and a storage element. Dr. Smith has integrated the DAIM with a conversational interface on his computer.
1. Patient Consultation: Sarah, a 45-year-old woman, visits Dr. Smith's clinic for a routine check-up. Dr. Smith initiates the consultation and activates the PMS system to assist him.
2. Conversation Capture: Dr. Smith starts the recording device, such as a digital transcription device, to capture the conversation between him and Sarah. This ensures that the entire consultation is recorded for further analysis.
3. Transcription: The system automatically transcribes the conversation using an automated speech recognition (ASR) technology. The recorded audio is converted into a textual transcription, providing a written record of the discussion.
4. Generating the PMS: The transcribed conversation is processed by the Diagnosis-AI Module (DAIM). The DAIM extracts clinically relevant information from the conversation, such as Sarah's symptoms, medical history, and any concerns she raises during the consultation.
5. PMS Format: Dr. Smith prefers a template-filling format for the PMS. The DAIM uses this format to generate the personalized medical summary, filling in the relevant information extracted from the conversation.
6. Relevant Suggestions: As Dr. Smith reviews the PMS, the DAIM suggests potential diagnoses, treatment options, and follow-up actions based on the information extracted. The DAIM assists Dr. Smith in providing comprehensive care by suggesting further tests or referrals if necessary.
7. Integration with Conversational Interface: Dr. Smith interacts with the system through the conversational interface integrated with the DAIM. He can ask questions or seek additional information related to Sarah's case, and the DAIM responds accordingly.
8. Medical Record Storage: The system includes a Medical Record Storage Module (MRSM) where Dr. Smith's patient data, including the previously generated PMS and past practitioner-patient conversations, are stored. This ensures easy access to historical information for future reference and analysis.
9. Patient Engagement: Dr. Smith discusses the findings with Sarah, sharing the PMS summary and explaining the potential diagnoses, treatment options, and recommended follow-up actions. The personalized nature of the summary helps Sarah understand her health status and the next steps involved in her care.

By using the personalized medical summary system, Dr. Smith can efficiently capture and transcribe practitioner-patient conversations, extract relevant information, generate a comprehensive summary, and receive valuable suggestions for diagnosis and treatment. The system enhances the quality of care provided by Dr. Smith, improves patient engagement, and enables efficient documentation and information retrieval for future consultations.

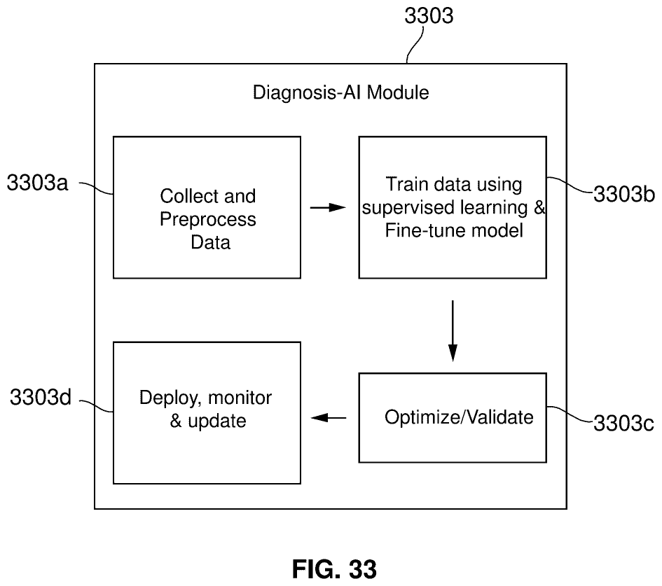

Now with reference to FIG. 33, in an embodiment of the invention the DAIM 3303 is generated by collecting and preprocessing a medical/dental data 3303a, using the preprocessed medical/dental data to train the DAIM by supervised fine-tuning 3303b, optimizing and validating the trained DAIM using reinforcement learning 3303c and deploying and updating the DAIM on at least one of a new patient data, and a new practitioner diagnosis/treatment data to evaluate DAIM performance 3303d.

In an embodiment of the invention, the collection of medical/dental data can be collected from at least one of, electronic health records, clinical trials, research studies, practitioner-patient conversations, medical history, symptoms, diagnoses, treatments, and other relevant information. The data is further pre-processed to remove any personal identifiable or sensitive information-anonymize data. Additionally, in an embodiment, a separate dataset of labeled diagnoses that correspond to the symptoms and medical information discussed in the conversations is also collected. Further yet, the conversation data is annotated with relevant labels or tags to identify important sections or entities within the conversations, such as symptoms, diagnoses, medications and patient information to identify key information accurately. For example—marking a symptom like “headache” as a symptom entity and labeling a diagnosis like “migraine” as a diagnosis entity.

Further yet, in an embodiment for training data preparation begins by splitting the annotated conversation dataset into training, validation, and testing sets and allocating a portion of the diagnosis dataset for training the Diagnosis AI-module (DAIM). Further yet, the DAIM training is carried out using the labeled diagnosis dataset by taking symptoms and other medical information as input and predicting the corresponding diagnosis. The DAIM is trained to predict the diagnosis for a given piece of medical/dental data. The next step in an embodiment of the invention involves of optimizing and validating the trained DAIM using reinforcement learning. Reinforcement learning allows the DAIM to learn from its own experiences. The DAIM is given a series of tasks, such as diagnosis patients or generating treatment plans. The DAIM is rewarded for completing tasks correctly and penalized for completing tasks incorrectly. Further in an embodiment of the invention, once the DAIM has been optimized and validate, it is deployed on new patient data or practitioner diagnosis/treatment to improve its performance.

In yet another embodiment of the invention, a PMS may be generated from practitioner-patient conversation, with or without a DAIM. A large language model LLM, for example—ChatGPT, Bard may be fine-tuned or custom built to extract clinically relevant information from the practitioner-patient conversation and directly rendered into a PMS.

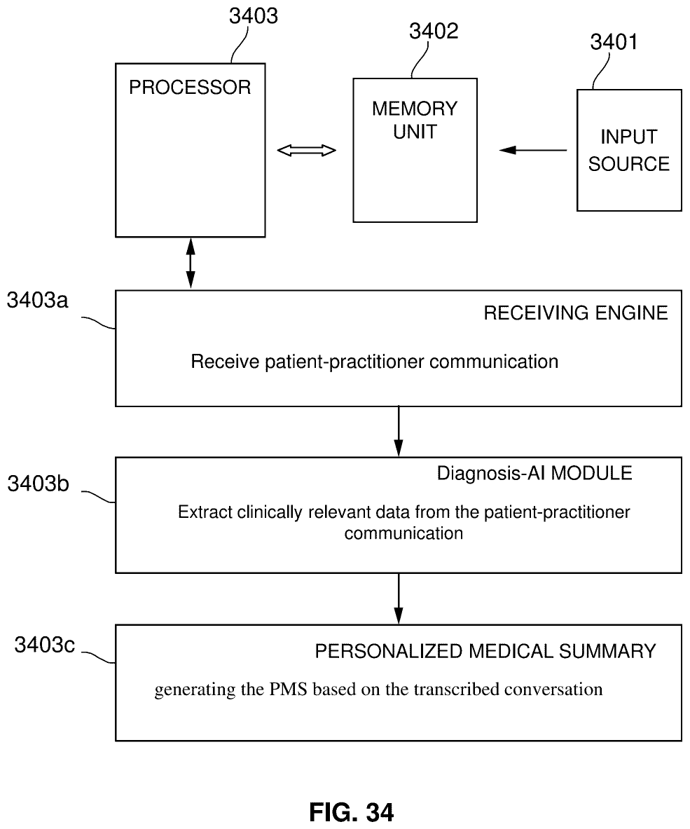

Now with reference to FIG. 34, in an embodiment of the invention, a system to generate a personalized medical summary (PMS) from a practitioner-patient conversation, comprising a processor 3403, a diagnosis-AI module (DAIM) 3403b, a non-transitory storage element coupled to the processor over a network, encoded instructions stored in the non-transitory storage element, wherein the encoded instructions when implemented by the processor 3403, configure the system to capture a conversation between the practitioner and the patient by the receiving engine 3403a, transcribe the conversation between the practitioner and the patient and generate the PMS 3403c for the patient based on the transcribed conversation via the DAIM. The practitioner or patient interacts with the system via a DAIM 3403b, which is configured to receive a practitioner-patient conversation as the input 3401. This innovative system uses generative AI, principles of transformer architecture, and reinforcement learning to extract clinically relevant data from the practitioner-patient conversation to generate a personalized medical summary. The DIAM may utilize transformer architecture, akin to that of the GPT-4 model, which uses multiple layers with self-attention and feedforward networks to understand the practitioner-patient conversation. The foundation of the system is the DAIM module 3403b, which is effectively designed to function like a transformer-based model. It accepts practitioner-patient conversations, similar to the input sentences a language model would take, then applies self-attention and feedforward layers to generate relevant code or scripts. In this context, self-attention layers help the DAIM module 3403b identify the relative importance of each word or phrase in the conversation. This allows the module to understand what features of the input should be given higher priority while generating the code. The feedforward layers, on the other hand, apply non-linear transformations, enabling the extraction of complex features and relationships within the input data. Using these capabilities, the DAIM module 3403b, akin to a transformer model during training, generates a script based on the context and relationships of words in the conversations between the practitioner and patient. The script can be seen as the “predicted output” of the DIAM module 3403b. The generated script instructs the effects engine on how generate personalized medical summary unique to a patient.

This whole process of using generative AI to capture practitioner-patient conversation, transcribe conversation and generate a PMS can be seen as a form of reinforcement leaning from human feedback. In reinforcement learning from human feedback, the AI system is trained and fine-tuned based on feedback from human trainers who can provide rewards or penalties based on the AI's performance. In this case, the “reward” and “penalty” comes from how well the PMS—unique to a patient diagnosis is generated from the captured practitioner-patient conversations. If the PMS matches the practitioner's expectations, this could be seen as a “reward” promoting the actions taken by the DAIM module 3403b. Conversely, if the PMS generated fails to match the practitioner's expectations—not unique to a particular patient diagnosis, this could be seen as a “penalty”, suggesting that the DIAM module 3403b needs to adjust its code generation process.

The system starts with the DAIM module 3403b, serving as the heart of this system, which receives practitioner-patient conversations. Similar to the large language model, the DAIM module 3403b analyzes the practitioner-patient conversations using self-attention and feedforward layers to generate an appropriate code or script. The self-attention layer weighs the importance of words or phrases in the conversation, prioritizing essential elements, while the feedforward layer executes non-linear transformations to extract complex features from the input conversations. This outputted code or script generates a PMS (may or may not suggest a diagnosis or treatment) unique to a patient. This process is similar to the learning and predicting process of the transformer model, where the system analyzes the input (practitioner-patient conversation), generates a prediction (code or script) and then applies the prediction (personalized medical summary). The performance of this entire process can be optimized using reinforcement learning from human feedback (RLHF). User feedback and reactions to the rendered effects can be used as signals to “reward” or “penalize” the system's performance, subsequently fine-tuning the operations of the DAIM modules.

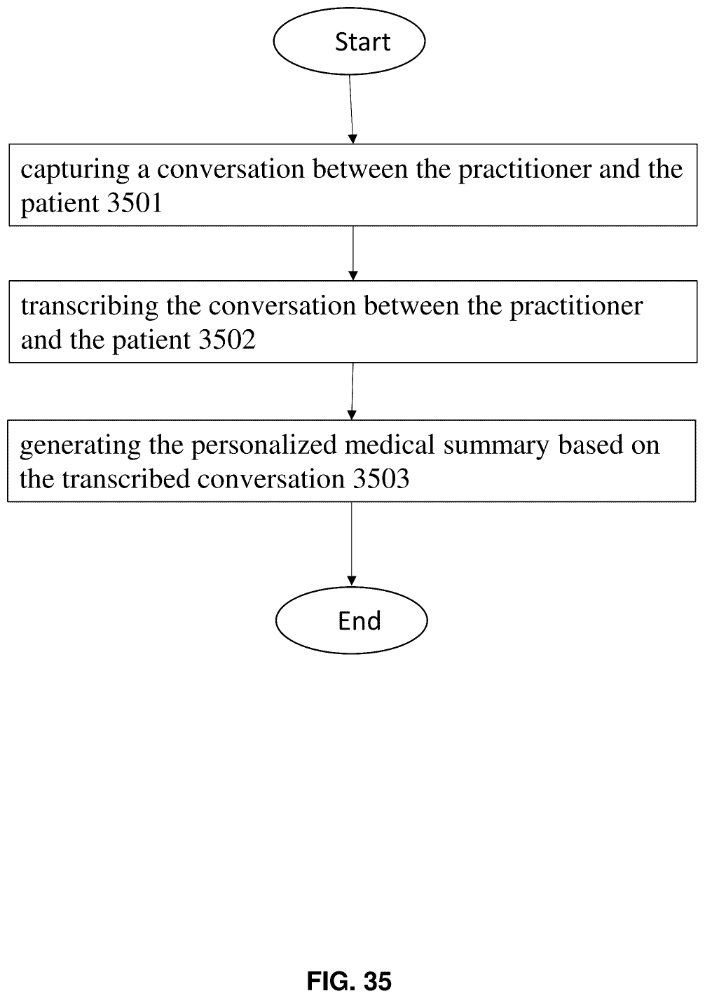

Now with reference to FIG. 35, in yet another embodiment of the invention, a method to generate a personalized medical summary (PMS) from practitioner-patient communication comprising capturing a conversation between the practitioner and the patient 3701, transcribing the conversation between the practitioner and the patient 3702 and generating the PMS for the patient based on the transcribed conversation, wherein the PMS is generated by a dental diagnosis module (DDM) using diagnosis-AI module (DAIM) 3703. In an embodiment of the invention, further comprising a recording device for capturing practitioner-patient conversations wherein, the recording device is at least one of voice recorders, smart phones, smart & digital devices, microphones, cameras, audio or video recorder, PC, and digital transcription devices. Further yet, the practitioner-patient conversation is transcribed into a textual transcription using an automated speech recognition (ASR) wherein, the ASR at least one of, an off-the shelf, custom-built or a third-party service. Further in an embodiment of the invention, extracting clinically relevant information by the diagnosis AI-module (DAIM) using at least one of a general-purpose large language model (LLM), a fine-tuned LLM trained for medical conversation or a custom-built LLM and rendering the PMS wherein, the PMS is rendered using at least one of a template-filling, do-by-example, and free-form summary format. In an alternative embodiment, the DAIM module may use large language models or generative AI foundational principles such as principles of transformer architecture, and reinforcement learning to generate a PMS based on transcribed practitioner-patient conversations. Further yet, in an embodiment of the invention, integrating the diagnosis AI-module (DAIM) with a at least one of a conversational interface, chatbot or a voice-based AI-assistant wherein, the DAIM integration is via by at least one of third-party API integration, file-based integration, screen scraping, and direct database integration.

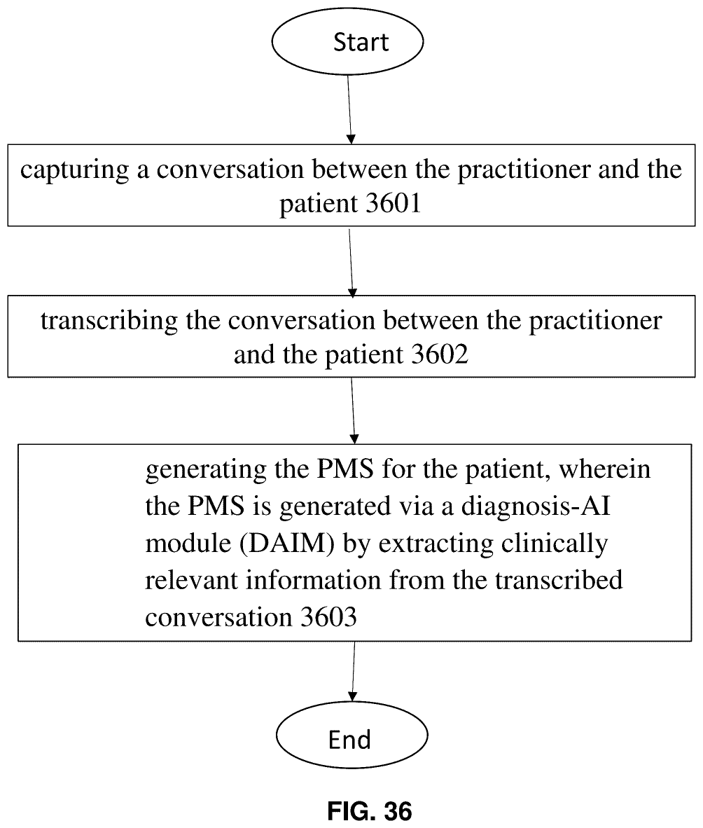

Now with reference to FIG. 36, in an embodiment of the invention, a method to generate a personalized medical summary (PMS) from practitioner-patient communication, comprising capturing a conversation between the practitioner and the patient, transcribing the conversation between the practitioner and the patient and generating the PMS for the patient, wherein the PMS is generated via a diagnosis-AI module (DAIM) by extracting clinically relevant information from the transcribed conversation.

Further yet, in an embodiment of the invention once, the patient responds to the first set of queries generated by the DAIM, the DAIM generates a second set of queries or a dental diagnosis to enhance practitioner-patient communication.

Further yet, in an embodiment of the invention, a network diagram of the practitioner-patient communication system. The networked environment includes a patient input, a practitioner prompt and a dental diagnosis module. The patient input and practitioner prompt and the DAIM are communicatively coupled through a network. Typically, the DAIM generates a query based on the patient input and a practitioner prompt to enhance patient-practitioner communication.

In an exemplary embodiment, the network facilitates communication between a DAIM, patient data input, and the practitioner prompt with the DAIM featuring collection and preprocessing of data, training using supervised fine-tuning, optimization and validation and deploying, monitoring and updating of the DAIM. The patient input data retrieves patient data and information related to healthcare interactions and visits. Further yet, the practitioner prompt includes for visually-aided notes, diagnosis, recommendations, prescribed treatment, interactive maps and timelines and the DAIM-rendered queries, with the network facilitating this communication. The DAIM processes the patient input data as well as the data provided by the practitioner and generates practitioner queries for the patients. Once, the patient responds to the first set of queries generated by the DAIM, the DAIM generates a second set of queries or a dental diagnosis to enhance practitioner-patient communication. An intuitive input/output system allows practitioners and patients to navigate the query/diagnosis modules. In another embodiment of the invention, the system may also incorporate a UX/UI to visualize a diagnosis report or queries, which provides a quick and efficient way for practitioners to input patient data and patients to respond to a query for an enhanced practitioner-patient communication. Examples of patient data inputs, but not limited to, symptom description, medical history, lifestyle factors, medication and supplement use, fears/concerns of diagnosis/treatments, expectations/goals regarding diagnosis/treatment. Further yet, patients may provide data via a real-time uploading of body worn devices typically embedded/equipped with one or more motion sensors, physiological sensors and environmental sensors. Examples of these sensors include, but are not limited to accelerometers, gyroscopes, inclinometers, geomagnetic sensors, global positioning systems, impact sensors, microphones, cameras, heart rate monitors, pulse oximeters, blood alcohol monitors, respiratory rate sensors, transdermal sensors, galvanic skin response (GSR) sensors and electromyography (EMG) sensors. In an embodiment of the present invention, the data captured by the one or more sensors is sent to the DAIM and, or the patient input through the network. Typically, the body worn device is worn on one or more body parts of the patient, such as wrist, waist, neck, arm, leg, abdomen, chest, thigh, head, ear and fingers. Further, the body worn device may be a wristband, a watch, an armband, a necklace, a headband, an earring, a waist belt and a ring. The body worn device communicates with the mobile communication device (including, but not limited to, a smartphone, a tablet, a personal digital assistant (PDA), thin-client, and a mobile phone) over a short-range wireless communication medium. Examples of the short-range wireless communication medium include Bluetooth, ZigBee, Infrared, Near Field Communication (NFC) and Radio-frequency identification (RFID). Additionally, examples of practitioner-fed patient data include, but not limited to, diagnosis, treatment, planning and follow-up of dental/medical procedures.

In a continuing reference, the network may be any suitable wired network, wireless network, a combination of these or any other conventional network, without limiting the scope of the present invention. Few examples may include a LAN or wireless LAN connection, an Internet connection, a point-to-point connection, or other network connection and combinations thereof. The network may be any other type of network that is capable of transmitting or receiving data to/from host computers, personal devices, mobile phone applications, video/image capturing devices, video/image servers, or any other electronic devices. Further, the network is capable of transmitting/sending data between the mentioned devices. Additionally, the network may be a local, regional, or global communication network, for example, an enterprise telecommunication network, the Internet, a global mobile communication network, or any combination of similar networks. The network may be a combination of an enterprise network (or the Internet) and a cellular network, in which case, suitable systems and methods are employed to seamlessly communicate between the two networks. In such cases, a mobile switching gateway may be utilized to communicate with a computer network gateway to pass data between the two networks. The network may include any software, hardware, or computer applications that can provide a medium to exchange signals or data in any of the formats known in the art, related art, or developed later.

Further yet in an embodiment of the invention, a system and method for delivering an enhanced practitioner-patient communication, including a memory system for storing dental/medical data to include, but not limited to, 1. Patient demographics such as name, age, gender, address, contact details, and insurance information, 2. Medical history to include past and current medical conditions, previous surgeries, allergies, medications, and family medical history, 3. Dental records to include information on dental examinations, diagnoses, treatments, and outcomes, as well as dental x-rays, CBCT scans, impressions, and photographs, 4. Vital signs to include measurements such as blood pressure, heart rate, respiratory rate, temperature, and oxygen saturation, 5. Laboratory results to includes blood tests, urine tests, and other diagnostic tests, which provide information on various health parameters, such as blood glucose levels, cholesterol levels, and liver function, 6. Imaging studies to include radiology reports, such as X-rays, CT scans, MRI scans, and ultrasound reports, which help in diagnosing and monitoring various conditions, 7. Prescription and medication information to include information on prescribed medications, dosage, frequency, and duration of use, 8. Surgical and procedural data to include information on surgeries, procedures, and interventions performed, including details such as date, type of procedure, surgical notes, and anesthesia used and, 9. Practitioner progress notes to include documentation of healthcare providers' observations, assessments, diagnoses, and treatments during patient visits, including dental and medical progress notes. The processor system is configured to process the stored data and generate a query for a patient via the DAIM.

The input system is configured to receive patient data, practitioner prompts and information related to present/past visits. The output system is configured to present a diagnosis/treatment report. The UX/UI system is configured to provide an intuitive and interactive interface for the patients and practitioners to navigate through the queries which enables the patient to make an informed decision regarding the treatment/planning for the received diagnosis.

In an embodiment of the present invention involves, a system and method to deliver a practitioner-patient communication, said method comprising: receiving inputs from patient and a practitioner prompt, generating a practitioner first query for a patient based on the received patient data and the practitioner prompt and generating at least one of a practitioner second query or a diagnosis for the patient based on the patient response to the practitioner first query to enhance practitioner-patient communication. Additionally, the practitioner first query for the patient is based on the received patient data and the practitioner prompt, wherein the query is generated a dental diagnosis module (DAIM). The DAIM is generated by collecting and preprocessing patient data, using the preprocessed data to train the DAIM by supervised fine-tuning, optimizing the trained DAIM using reinforcement learning, testing and validating the DAIM on at least one of a new patient data, and a new practitioner diagnosis/treatment data to evaluate the DAIM performance and integrating and deploying the DAIM with a at least one of a conversational interface, chatbot or a voice-based AI-assistant (not shown). Further yet, the DAIM integration with a conversational interface, chatbot or a voice-based AI-Assistant may be accomplished via at least one of third-party API integration, file-based integration, screen scraping, and direct database integration.

A third-party API integration involves integration of the DAIM with a third-party API, for example, chatGTP which provides the necessary interface for the chatbot to communicate with the DAIM module. The chatGTP may additionally provide natural language processing and speech recognition capabilities as well as standard communication protocols or a custom interface. Alternatively, file-based integration involves exporting the patient data from the DAIM into a file format that can be consumed by the conversational interface. The conversational interface can then read the patient data from the file and respond accordingly. The file format should be agreed upon by both the systems and should be in a format that is easy to parse. Further yet, screen scraping involves using software to extract information from the DAIM's user interface. The chatbot or AI assistant can then use this information to provide a diagnosis or other information to the patient. Furthermore, direct database integration involves integrating the DAIM with the chatbot or AI assistant's database. The DAIM module may write information to the database, which the chatbot or AI assistant can then read and use in the conversation. Further yet, in an embodiment of the invention the DAIM may be integrated with a dialog flow API which uses machine learning algorithms to analyze and interpret patient inputs and practitioner prompts and then generates appropriate responses based on the context of the conversation.

Yet in another embodiment of the invention, consider a scenario wherein Jane, the patient is suffering from a tooth pain and decides to visit the dentist Jacob at his clinic. Upon arrival, Jane enters her current condition and medications into the practitioner-patient system. Jacob performs the oral cavity examination of Jane and concludes—a significant tooth cavity which, he then enters into the practitioner-patient system. Upon receiving a prompt from Jacob, the dental diagnosis module then generates a query for Jane based on Jacob's examination and Jane's inputs—“Have you noticed any other symptoms such as fever, or sensitivity to hot or cold foods? Have you recently experienced any trauma or injury to the affected tooth?” Jane's responds to the query—She had experienced sensitivity and severe discomfort to the affected tooth when she chewed an almonds the prior evening. Based on Jane's response, past treatments, medical history, and Jacob's oral cavity examination, the dental diagnosis module further generates a diagnosis—deep dental cavity. Upon receiving the diagnosis, Jane further askes—“Will I need a root canal or a tooth extraction?”. Seeing a flustered Jane, Jacob prompts the system to further expand on the deep dental cavity diagnosis—Jacob's enters his response to the system—a tooth extraction. The dental diagnosis module explains to Jane of why Jacob is recommending a tooth extraction because, A) Severe decay of the tooth, B) A tooth fracture was caused because of Jane chewing on almonds.

The DAIM may also provide Jane with information about what to expect during and after the tooth extraction procedure, including the steps involved, potential risks and complications, and post-operative care instructions. Further yet, the DAIM may also provide information about the potential risks and complications of the procedure, such as bleeding, infection, or damage to adjacent teeth or structures. Jane may be advised to avoid certain activities, such as smoking or drinking through a straw, to minimize the risk of complications. Additionally, the DAIM may recommend that Jane consider a replacement option for the extracted tooth, such as a dental implant, bridge, or denture. The DAIM may provide information about the different types of replacement options and what to expect during the restoration process. Overall, the DAIM will provide Jane with important information and guidance about her tooth extraction and what to expect before, during, and after the procedure. This can help her feel more prepared and confident about her treatment plan-thus, improving practitioner-patient communication.

Further yet in an embodiment of the invention the patient data is at least one of current patient condition, patient dental/medical disease history, physical and mental health, past dental/medical treatments, X-rays/scans, medical complaints and list of medication. Additionally, the dental query is based on at least one of a diagnosis, treatment, planning, and follow-up of the dental procedures for the patient based on the practitioner fed patient data via a practitioner prompt.

Further yet, in an embodiment of the invention, the process of generating a DAIM using supervised fine-tuning and reinforcement learning can be broken down into several steps:

Define the problem: Clearly define the problem you are trying to solve. In this case, the problem is to generate a dental diagnosis module that can accurately predict the outcomes of various dental treatments to enhance practitioner-patient communication.

Collect data: Collect data that can be used to train and validate the dental diagnosis module. This data should include information about patient demographics, medical history, dental history, and treatment outcomes.

Preprocess data: Clean and preprocess the data to ensure it is ready for training. This may involve removing duplicates, filling in missing values, and converting categorical variables to numerical values.

Train the supervised learning module: Use a supervised learning algorithm, such as a neural network or decision tree, to train the dental diagnosis module on the preprocessed data. The module will learn to predict treatment outcomes based on patient information.

Fine-tune the module: Use reinforcement learning to fine-tune the dental diagnosis module. Reinforcement learning involves using rewards and punishments to guide the module's behavior. For example, the module may receive a reward for accurately predicting a treatment outcome, and a punishment for making an inaccurate prediction.

Validate the module: Use a validation dataset to evaluate the performance of the dental diagnosis module. This will help ensure that the module is generalizing well to new data.

Deploy the module: Once the dental diagnosis module has been trained and validated, it can be deployed in a real-world setting to assist dental practitioners in making treatment decisions.

Monitor and update the module: It is important to monitor the performance of the dental diagnosis module over time and update it as new data becomes available or treatment protocols change. This will help ensure that the module remains accurate and effective.

To generate a query using a dental diagnosis module (DAIM) optimized to mimic practitioner preferences through a combination of supervised fine-tuning and reinforcement learning, and performed by a chatbot or AI assistant, the following steps can be taken:

Data Collection: Gather a dataset of dental policies, procedures, and practitioner preferences. This can include a combination of published policies, practitioner guidelines, and real-world practitioner behavior data.

Pre-processing: Pre-process the dataset to ensure that it is clean and suitable for training. This may involve data cleaning, normalization, and feature engineering.

Module Training: Train the DAIM module using supervised learning to initially teach the module how to mimic practitioner preferences based on the gathered dataset. The module can then be further optimized through reinforcement learning to learn from interactions with real-world practitioners or patients.

Fine-tuning: Fine-tune the module using supervised learning with new data to update and improve the module's accuracy and ability to mimic practitioner preferences.

Integration: Integrate the DAIM module with a chatbot or AI assistant to enable it to generate queries based on input from patients or practitioners.

Query Generation: Use the DAIM module to generate queries based on the input provided to the chatbot or AI assistant. The module will use its learned knowledge and understanding of practitioner preferences to generate the most appropriate and relevant queries.

Validation: Validate the generated queries to ensure that they are accurate and relevant to the input provided.

Iteration: Iterate and improve the module and query generation process by collecting feedback from practitioners and patients and incorporating this into the training and fine-tuning process.

By following these steps, a dental diagnosis module (DAIM) optimized to mimic practitioner preferences through a combination of supervised fine-tuning and reinforcement learning can be trained and integrated with a chatbot or AI assistant to generate queries that are accurate and relevant to the input provided.

Further yet, in an embodiment of the invention, at least one query is generated by training a dental diagnosis module (DAIM) optimized to mimic the practitioner preferences by combining supervised fine-tuning and reinforcement learning and performed by at least one of a chatbot, or an AI-assistant. Generating a query for a dental diagnosis module (DAIM) that mimics practitioner preferences can be achieved through a combination of supervised fine-tuning and reinforcement learning.

For example, in an embodiment of the invention, supervised fine-tuning may involve training the DAIM on a dataset of examples that have been labeled by practitioners to reflect their preferences. The DAIM learns to identify patterns and features in the examples, which it can then use to generate queries that closely mimic the preferences of the practitioners. The labeled dataset is created by experts in the dental or medical field or by collecting data from at least one of, but not limited to, electronic health records, surveys, scans, X-rays, images or other sources.

Further yet, in an embodiment of the invention, reinforcement learning involves training the DAIM to maximize a reward function based on the preferences of practitioners. The reward function provides a way to evaluate the quality of the queries generated by the DAIM. The DAIM receives feedback in the form of a reward signal, which is used to adjust its behavior and generate better queries in the future. The reward signal can be based on various factors, such as but not limited to, the accuracy of the diagnosis, the speed of response, or the level of patient satisfaction.

Once the DAIM is trained, it can be integrated into a chatbot or AI-assistant to generate queries in real-time. The chatbot or AI-assistant can interact with patients, gather information about their symptoms and medical history, and use the DAIM to generate queries that reflect the preferences of practitioners. The chatbot or AI-assistant can also use the reinforcement learning approach to continuously improve the quality of the queries over time, based on feedback from practitioners and patients.

The figures illustrate the architecture, functionality, and operation of possible implementations of systems, methods and computer program products according to various embodiments of the present invention. It should also be noted that, in some alternative implementations, the functions noted/illustrated may occur out of the order noted. For example, two blocks shown in succession may, in fact, be executed substantially concurrently, or the blocks may sometimes be executed in the reverse order, depending upon the functionality involved.

Since various possible embodiments might be made of the above invention, and since various changes might be made in the embodiments above set forth, it is to be understood that all matter herein described or shown in the accompanying drawings is to be interpreted as illustrative and not to be considered in a limiting sense. Thus, it will be understood by those skilled in the art that although the preferred and alternate embodiments have been shown and described in accordance with the Patent Statutes, the invention is not limited thereto or thereby.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Additionally, it is to be understood that references to anatomical structures may also assume image or image data corresponding to the structure. For instance, extracting a teeth arch translates to extracting the portion of the image wherein the teeth arch resides, and not the literal anatomical structure.

Some portions of embodiments disclosed are implemented as a program product for use with an embedded processor. The program(s) of the program product defines functions of the embodiments (including the methods described herein) and can be contained on a variety of signal-bearing media. Illustrative signal-bearing media include, but are not limited to: (i) information permanently stored on non-writable storage media (e.g., read-only memory devices within a computer such as CD-ROM disks readable by a CD-ROM drive); (ii) alterable information stored on writable storage media (e.g., floppy disks within a diskette drive or hard-disk drive, solid state disk drive, etc.); and (iii) information conveyed to a computer by a communications medium, such as through a computer or telephone network, including wireless communications. The latter embodiment specifically includes information downloaded from the Internet and other networks. Such signal-bearing media, when carrying computer-readable instructions that direct the functions of the present invention, represent embodiments of the present invention.

In general, the routines executed to implement the embodiments of the invention, may be part of an operating system or a specific application, component, program, module, object, or sequence of instructions. The computer program of the present invention typically is comprised of a multitude of instructions that will be translated by the native computer into a machine-accessible format and hence executable instructions. Also, programs are comprised of variables and data structures that either reside locally to the program or are found in memory or on storage devices. In addition, various programs described may be identified based upon the application for which they are implemented in a specific embodiment of the invention. However, it should be appreciated that any particular program nomenclature that follows is used merely for convenience, and thus the invention should not be limited to use solely in any specific application identified and/or implied by such nomenclature.

The present invention and some of its advantages have been described in detail for some embodiments. It should also be understood that various changes, substitutions and alterations can be made herein without departing from the spirit and scope of the invention as defined by the appended claims. An embodiment of the invention may achieve multiple objectives, but not every embodiment falling within the scope of the attached claims will achieve every objective. Moreover, the scope of the present application is not intended to be limited to the particular embodiments of the process, machine, manufacture, and composition of matter, means, methods and steps described in the specification. A person having ordinary skill in the art will readily appreciate from the disclosure of the present invention that processes, machines, manufacture, compositions of matter, means, methods, or steps, presently existing or later to be developed are equivalent to, and fall within the scope of, what is claimed. Accordingly, the appended claims are intended to include within their scope such processes, machines, manufacture, compositions of matter, means, methods, or steps.

We claim:

1. A method to generate a personalized medical summary (PMS) from a practitioner-patient conversation, said method comprising:
capturing a conversation between a practitioner and a patient;
transcribing the conversation between the practitioner and the patient; and
generating the PMS based on the transcribed conversation.

2. The method of claim 1, further comprising a recording device for capturing practitioner-patient conversation.

3. The method of claim 2, wherein the recording device is at least one of voice recorders, smart phones, smart & digital devices, microphones, cameras, audio or video recorder, PC, and digital transcription devices.

4. The method of claim 1, further comprising transcribing the practitioner-patient conversation into a textual transcription using an automated speech recognition (ASR).

5. The method of claim 4, wherein the ASR is at least one of, off-the shelf, custom-built or a third-party service.

6. The method of claim 1, further comprising extracting clinically relevant information by a diagnosis AI-module (DAIM) using at least one of a general-purpose large language model (LLM), a fine-tuned LLM trained for medical conversation or a custom-built LLM and rendering the PMS.

7. The method of claim 6, wherein the PMS is rendered using at least one of a template-filling, do-by-example, and free-form summary format.

8. The method of claim 6, further comprising integrating the diagnosis AI-module (DAIM) with a at least one of a conversational interface, chatbot or a voice-based AI-assistant.

9. The method of claim 6, wherein the DAIM integration is via by at least one of third-party API integration, file-based integration, screen scraping, and direct database integration.

10. A system to generate a personalized medical summary (PMS) from a practitioner-patient conversation, comprising:
a processor;
a diagnosis-AI module (DAIM);
a non-transitory storage element coupled to the processor over a network;
encoded instructions stored in the non-transitory storage element, wherein the encoded instructions when implemented by the processor, configure the system to:
capture a conversation between a practitioner and a patient;
transcribe the conversation between the practitioner and the patient; and
generate the PMS for the patient based on the transcribed conversation via the DAIM.

11. The system of claim 10, further comprising a recording device to capture practitioner-patient communications.

12. The system of claim 11, wherein the recording device is at least one of voice recorders, smart phones, smart & digital devices, microphones, cameras, audio or video recorder, and digital transcription devices.

13. The system of claim 12, further comprising the practitioner-patient conversation transcribed into a textual transcription via an automated speech recognition (ASR).

14. The system of claim 13, wherein the automated speech recognition is at least one of, an off-the shelf, custom-built or a third-party service.

15. The system of claim 13, wherein automated speech recognition is performed by at least one of acoustic modeling-based ASR or neural network-based ASR.

16. The system of claim 10, wherein the diagnosis-AI module (DAIM) extracts clinically relevant information using at least one of a general-purpose large language model (LLM), a fine-tuned LLM trained for medical conversation or a custom-built LLM to render the PMS.

17. The system of claim 16, wherein the PMS is rendered using at least one of a template-filling, do-by-example, and free-form summary format.

18. The system of claim 16, further comprising integrating the DAIM with a at least one of a conversational interface, chatbot or a voice-based AI-assistant.

19. The system of claim 16, wherein the DAIM integration is via by at least one of third-party API integration, file-based integration, screen scraping, and direct database integration.

20. The system of claim 16, wherein the DAIM suggests relevant information to the practitioner related to at least one of, potential diagnosis, treatment, planning, follow-up and communication with the patient.

21. The system of claim 10, further comprising a medical record storage module (MRSM) to record and save at least one of, patient data, previously generated PMS and past practitioner-patient conversations.

22. The system of claim 21, wherein the patient data is at least one of current patient condition, patient dental/medical disease history, physical and mental health, past dental/medical treatments, X-rays/scans, medical complaints and list of medication.

23. The system of claim 21, wherein MRSM is securely encrypted to ensure the privacy and confidentiality of patient data.

24. The system of claim 21, further comprising a search functionality within the MRSM to retrieve and display relevant patient information during the practitioner-patient conversation.

25. The system of claim 21, wherein the MRSM is integrated with electronic health record (EHR) systems to synchronize and update patient data.

26. A method to generate a personalized medical summary (PMS) from practitioner-patient communication, said method comprising:
capturing a conversation between the practitioner and the patient;
transcribing the conversation between the practitioner and the patient; and
generating the PMS for the patient, wherein the PMS is generated via a diagnosis-AI module (DAIM) by extracting clinically relevant information from the transcribed conversation.
